(19) United States
(12) Patent Application Publication
(10) Pub. No.: US 2026/0209724 A1
(43) Pub. Date: Jul. 23, 2026

(54) NOVEL PEPTIDE DERIVED FROM NDST3 OR NDST4 HAVING DEACETYLASE OR SULFOTRANSFERASE ACTIVITY

(71) Applicant: KOREA INSTITUTE OF SCIENCE AND TECHNOLOGY, Seoul (KR)

(72) Inventors: Sang Chul SHIN, Seoul (KR); Sang Soo CHOI, Seoul (KR); Jinju SEO, Seoul (KR); Seung Eun LEE, Seoul (KR); Yubin HONG, Seoul (KR); Jung Min YOON, Seoul (KR); Hyoyong PARK, Seoul (KR)

(21) Appl. No.: 19/201,625

(22) Filed: May 7, 2025

(30) Foreign Application Priority Data
Jan. 21, 2025 (KR) 10-2025-0009051

(51) Int. Cl. C12N 9/10

(52) U.S. Cl. CPC C12N 9/13; C12Y 208/02008

(57) ABSTRACT

The present disclosure relates to peptides derived from NDST3 or NDST4 with deacetylase or sulfotransferase activity, a method for producing the same, and uses thereof. The present disclosure was accomplished by identifying a domain having deacetylase or sulfotransferase activity in the NDST3 and NDST4 enzymes, producing a water soluble peptide including the amino acid sequence of said domain, and determining the deacetylase or sulfotransferase of said peptide. It is expected that the peptide of the present disclosure will be used as a platform for developing protein drugs or inhibitors thereof utilizing NDST3 and NDST4 proteins.

### CROSS-REFERENCE TO RELATED APPLICATION(S)

This application claims the benefit of Korean Patent Application No. 10-2025-0009051, filed on Jan. 21, 2025, at the Korean Intellectual Property Office, the entire disclosure of which is incorporated herein by reference for all purposes.

### BACKGROUND

### 1. Field

The present disclosure relates to peptides derived from NDST3 or NDST4 with deacetylase or sulfotransferase activity, a method for producing the same, and uses thereof. The present disclosure was accomplished by identifying a domain having deacetylase or sulfotransferase activity in the NDST3 and NDST4 enzymes, producing a water soluble peptide including the amino acid sequence of said domain, and determining the deacetylase or sulfotransferase of said peptide.

### 2. Description of Related Art

NDST3 and NDST4 are members of the NDST family, and are enzymes that play an important role in the biosynthesis of heparan sulfate proteoglycans (HSPGs). NDST3 and NDST4 regulate the chemical modification and structural diversity of HSPGs to perform essential functions in cell-to-cell signaling, cell growth, and tissue development, thereby affecting neural development, neural regeneration, and pathological mechanisms in certain diseases.

NDST3 is highly expressed in specific tissues or cell types during the early embryonic stage, and its role is particularly prominent in the nervous system. The known functions of NDST4 are as follows: 1) NDST3 regulates the transformation of HSPGs, which play an important role in the growth and differentiation of nerve cells. The structure of heparan sulfate, which is regulated by NDST3, plays an important role in neural development as well as regeneration after neural damage by facilitating the binding of certain growth factors and extracellular matrix proteins. 2) NDST3-modified HSPGs bind various growth factors, such as FGF and VEGF, to regulate cell signaling thereby enhancing cell proliferation, differentiation, and survival signaling. 3) NDST3 regulates the inflammatory response by adjusting the composition of HSPGs with regard to the activity of immune cells, thereby being involved in the movement, adhesion, and activation of immune cells, and may be an important target in inflammatory diseases and autoimmune diseases.

NDST3 plays a particularly important role in the nervous system, where it is essential for the growth of neurons, axon guidance, and synapse formation; and plays a key role in signaling between neurons to ensure that neurons grow and communicate with each other properly, thus playing an important role in regulating neural development and growth. Additionally, NDST3 participates in the regeneration process after nervous system damage, thereby being involved in promoting damaged nerve regeneration and protecting nerve cells from damage caused by external factors or stress. NDST3 also affects the process of amyloid plaque accumulation, which is the cause of Alzheimer's disease, when modifications or mutations occur therein. Therefore, NDST3 protein may provide therapeutic and neuroprotective effects for promoting nerve regeneration in spinal cord injury, traumatic brain injury, etc., and thus may be utilized as a protein therapeutic agent for overcoming various nervous system injuries or neurotoxic environments, and furthermore, it may be utilized as a drug discovery platform for targeting neurological diseases such as Alzheimer's disease and Parkinson's disease.

As such, NDST3 and NDST4 proteins play a role in the promotion of nerve regeneration and neuroprotection. However, despite the research findings on NDST3 and NDST4 reported so far, many of them have not been experimentally proven, and the mechanisms by which NDST3 and NDST4 directly regulate nerve cells to promote nerve regeneration or protect nerve are unknown. Therefore, NDST3 and NDST4 are attracting attention as novel targets for the treatment of neurological diseases, and the development of new drugs based on these enzymes as targets for innovative treatments for various neurological diseases such as neurological diseases, nerve damage, and developmental disorders has implications. However, research into developing therapeutic agents is yet insignificant. In the development of medicaments using the NDST3 and NDST4 mechanisms, which have not been fully elucidated to date, research on the signal transduction system that causes activation and inhibition, and exploration of the activating and inhibiting factors of NDST3 and NDST4 proteins, are tasks for the development of therapeutic agents for neurodevelopmental and neurological diseases.

After conducting extensive research to keep up with such demand, the present inventors cloned the functional portions of the NDST3 and NDST4 proteins, N-deacetylase and N-sulfotransferase, or a predetermined portion including each of the N-deacetylase and N-sulfotransferase domains, to obtain water soluble proteins, and confirmed that these soluble NDST3 and NDST4 proteins would be useful as a platform for developing protein drugs or inhibitors, thereby creating the present disclosure.

The Sequence Listing created on Aug. 8, 2025 with a file size of 39,936 Bytes and filed herewith in xml file format as the file entitled “Sequence Listing_457MH0110US.xml” is hereby incorporated by reference in its entirety.

### SUMMARY

A technical goal to be achieved by the present disclosure is to identify a deacetylase or sulfotransferase active domain in NDST3 and NDST4, and to provide a peptide including the domain derived from NDST3 or NDST4 as a deacetylase or sulfotransferase.

In addition, the present disclosure aims to provide a method for mass-producing the peptide in a water soluble form using Escherichia coli (E. coli), and to provide a use of the peptide for treating neurological diseases.

However, the technical goals of the present disclosure are not limited to those described above, and other goals not mentioned herein will be clearly understood by one of ordinary skill in the art from the following description.

In this specification, amino acid sequences are listed in order from N-terminus to C-terminus.

Human NDST3 protein consists of the amino acid sequence of SEQ ID NO: 1, and human NDST4 protein consists of the amino acid sequence of SEQ ID NO: 5.

The present inventors have identified that the domain exhibiting deacetylase activity in the NDST3 protein is located at positions 72 to 569 a.a., and that the domain exhibiting sulfotransferase activity is located at positions 569 to 873 a.a.

In addition, the present inventors have identified that the domain exhibiting deacetylase activity in the NDST4 protein is located at positions 72 to 569 a.a., and that the domain exhibiting sulfotransferase activity is located at positions 569 to 873 a.a.

Accordingly, the present disclosure provides a peptide having deacetylase activity.

The peptide having the deacetylase activity may include or consist of an amino acid sequence of SEQ ID NO: 3 or SEQ ID NO: 7. When the peptide having the deacetylase activity includes an amino acid sequence of SEQ ID NO: 3 or SEQ ID NO: 7, the peptide may include a tag sequence for purification at the N-terminus of SEQ ID NO: 3 or SEQ ID NO: 7, and totally the amino acids are no more than 800 a.a. long.

In addition, the present disclosure provides a peptide having sulfotransferase activity.

The peptide having the sulfotransferase activity may include or consist of the amino acid sequence of SEQ ID NO: 4 or SEQ ID NO: 8. When the peptide having the sulfotransferase activity includes an amino acid sequence of SEQ ID NO: 4 or SEQ ID NO: 8, the peptide may include a tag sequence for purification at the N-terminus of SEQ ID NO: 4 or SEQ ID NO: 8, and totally the amino acids are no more than 800 a.a. long.

The amino terminus of the peptide of the present disclosure may be bound with a protecting group such as an acetyl group, a fluorenyl methoxy carbonyl group, a formyl group, a palmitoyl group, a myristyl group, a stearyl group, and polyethylene glycol (PEG), and the carboxy terminus of the peptide may be modified with a hydroxy group (—OH), an amino group (—NH₂), an azide (—NHNH₂), or the like. In addition, the terminal of the peptide or the R-group of the amino acid of the present disclosure may be bound with fatty acids, oligosaccharide chains, all nanoparticles (gold particles, liposomes, heparins, hydrogels, etc.), amino acids, carrier proteins, etc. The amino acid modifications described above serve to improve the potency and stability of the peptide of the present disclosure. As used herein, term “stability” refers not only to in vivo stability but also to storage stability (including storage stability when stored at room temperature, stored refrigerated, and stored frozen).

Each peptide constituting the peptide of the present disclosure may have one or more amino acid sequences substituted, modified, or deleted while maintaining its function.

In addition, the present disclosure provides a polynucleotide encoding a peptide having deacetylase activity or a peptide having sulfotransferase activity.

Furthermore, the present disclosure provides a peptide expression cassette including the polynucleotide.

In addition, the present disclosure provides a peptide expression vector having deacetylase activity or a peptide expression vector having sulfotransferase activity, which includes the expression cassette.

Additionally, the present disclosure provides a transformant which has been transformed with the vector.

In one embodiment of the present disclosure, the transformant may be E. coli.

As used herein, the term “polynucleotide” refers to a polymer of deoxyribonucleotides or ribonucleotides that exists in single-stranded or double-stranded form. The scope of this term encompasses RNA genomic sequences, DNA (gDNA and cDNA) and RNA sequences transcribed therefrom, and also includes analogues of natural polynucleotides unless otherwise noted.

In the present disclosure, a sequence encoding the peptide includes, without limitation, not only a sequence encoding the amino acids set forth by their respective sequence numbers, but also a sequence that exhibits 80% or more, particularly 90% or more, more particularly 95% or more, even more particularly 98% or more, and most particularly 99% or more homology to the above sequence, as long as it encodes a protein that exhibits substantially equivalent or corresponding efficacy to each of the above proteins. Further, it is apparent that an amino acid sequence having homology to the sequence above, part of which is deleted, modified, substituted, or added, is also within the scope of the present disclosure, as long as the resulting amino acid sequence has a biological activity substantially equivalent or corresponding to a combination protein of the respective sequence numbers mentioned herein.

As used herein, the term “homology” refers to a degree of similarity between nucleotide sequences encoding a protein or amino acid sequences constituting a protein. When the degree of homology is sufficiently high, the expression product and protein of the corresponding gene may have the same or similar activity. Additionally, homology may be expressed as a percentage based on the degree of matching with a given amino acid sequence or nucleotide sequence. In the present disclosure, a homologous sequence having the same or similar activity as a given amino acid sequence or nucleotide sequence is expressed as “% homology.” For example, the % homology may be confirmed by using standard software, specifically, BLAST 2.0, for calculating parameters such as score, identity, and similarity, or by comparing sequences via hybridization experiments performed under defined stringent conditions, and the appropriate hybridization conditions to be defined are within the scope of the relevant technology and may be determined by a method well known to those skilled in the art.

Further, in the polynucleotide encoding the peptide, various modifications may be made in the coding region without changing an amino acid sequence of the protein, in consideration of the codons preferred in an organism in which the protein is to be expressed, due to codon degeneracy. Thus, the polynucleotide may include, without limitation, any polynucleotide sequence encoding the respective proteins.

In addition, the polynucleotide includes not only a nucleotide sequence encoding the amino acid sequence of the peptide, but also a sequence complementary thereto. The complementary sequence includes not only a perfectly complementary sequence but also a substantially complementary sequence, which means a sequence that is capable of hybridizing under stringent conditions known in the art, for example, with the nucleotide sequence encoding the amino acid sequence of the peptide.

Hybridization requires that the two polynucleotides have complementary sequences, although mismatches between nucleotides are possible depending on the stringency of hybridization. The term “complementary” is used to describe the relationship between nucleotide bases that are capable of hybridizing with each other. For example, with respect to DNA, adenosine is complementary to thymine, and cytosine is complementary to guanine. Accordingly, the present disclosure may also include isolated polynucleotide fragments that are complementary to the entire sequence as well as substantially similar polynucleotide sequences.

As used herein, the term “expression vector” refers to a recombinant vector capable of expressing a protein of interest when introduced into a suitable host cell, and which includes essential regulatory elements operably linked to allow the expression of the gene insert. The term “operably linked” means that a nucleic acid expression control sequence and a nucleic acid sequence encoding a protein of interest are functionally linked such that they are able to perform their general function. The operative linkage with the recombinant vector may be prepared using genetic recombination techniques well known in the art, and site-specific DNA cleavage and ligation may be easily accomplished using enzymes generally known in the art.

Suitable expression vectors of the present disclosure may include expression control elements such as promoters, initiation codons, termination codons, polyadenylation signals, and enhancers, as well as signal sequences for targeting membrane or secretion. The initiation and termination codons are generally considered to be a part of the nucleotide sequence encoding an immunogenic target protein, and when the genetic construct is administered to a subject, they must be functional in the subject and must be in frame with the coding sequence. Common promoters may be constitutive or inducible, and include the lac, tac, T3 and T7 promoters for prokaryotic cells, and the simian virus 40 (SV40), mouse mammary tumor virus (MMTV) promoters, human immunodeficiency virus (HIV), e.g., the long terminal repeat (LTR) promoters of HIV, Moloney virus, cytomegalovirus (CMV), Epstein-Barr virus (EBV), Rous sarcoma virus (RSV) promoters, as well as the R-actin promoters; promoters derived from human hemoglobin, human muscle creatine and human metallothionein, for eukaryotic cells, but are not limited thereto.

Further, the expression vector may include a selectable marker for selecting a host cell containing the vector. The selectable markers are intended to be used for screening for cells transformed with a vector, and markers that confer a selectable phenotype, such as drug resistance, auxotrophy, resistance to cytotoxic agents, or expression of surface proteins, may be used. In an environment treated with a selective agent, only cells expressing the selectable marker will survive, making the transformed cells selectable. Additionally, if the vector is a replicable expression vector, it may include a replication origin, which is a specific nucleic acid sequence from which replication is initiated.

As recombinant expression vectors for inserting foreign genes, various types of vectors, such as plasmids, viruses, and cosmids, may be used. The types of recombinant vector are not particularly limited as long as they function to express a gene of interest and produce a protein of interest in various host cells, both prokaryotic and eukaryotic cells. However, specifically, a vector that may produce large quantities of foreign proteins in a form similar to their natural state while possessing a strong active promoter and a strong expression ability may be utilized.

To express the protein of the present disclosure, various combinations of hosts and vectors may be used. Expression vectors suitable for eukaryotic hosts include, but are not limited to, pCMV, pFLAG, pMYC, pHA, etc., and may also include expression control sequences derived from SV40, bovine papilloma virus, adenovirus, adeno-associated virus, cytomegalovirus, and retrovirus. Expression vectors that may be used in bacterial hosts include, but are not limited to, bacterial plasmids obtained from E. coli including pET21a, pET, pRSET, pBluescript, pGEX2T, pUC vectors, col E1, pCR1, pBR322, pMB9 or derivatives thereof; plasmids having a broader host range such as RP4; phage DNA, which may be exemplified by phage lambda derivatives such as λgt10, λgt11 or NM989; and other DNA phages such as M13 and filamentous single-stranded DNA phages. For yeast cells, 2° C. plasmid or its derivatives may be used, and for insect cells, pVL941, etc., may be used.

The cells, for example, eukaryotic cells, may be yeasts, fungi, protozoa, plants, higher plants and insects, or amphibian cells, or mammalian cells such as CHO, HeLa, HEK293, and COS-1, and may also be, for example, cultured cells (in vitro), graft cells and primary cell cultures (in vitro and ex vivo), and in vivo cells, as commonly used in the art, and also mammalian cells including human cells. Additionally, the organism may be a yeast, a mold, a protozoa, a plant, a higher plant, an insect, an amphibian, or a mammal.

However, in the present disclosure, the host cell may be a prokaryotic organism suitable for the purpose of mass producing the peptides described above, and may be E. coli, considering cost and time.

In addition, the present disclosure provides a method for mass producing a peptide having deacetylase activity or a peptide having sulfotransferase activity.

The method may include steps (1) through (5) listed below:
(1) preparing an expression vector including a gene encoding a peptide having deacetylase activity or a peptide having sulfotransferase activity;
(2) transforming E. coli with the expression vector;
(3) culturing the transformed E. coli;
(4) disrupting and centrifuging the transformed E. coli to obtain a cell precipitate; and
(5) purifying a peptide having deacetylase activity or a peptide having sulfotransferase activity from the cell precipitate.

The method may include steps (a) through (d) listed below:
(a) preparing E. coli transformed with an expression vector including a gene encoding a peptide having deacetylase activity or a peptide having sulfotransferase activity;
(b) culturing the transformed E. coli;
(c) disrupting and centrifuging the E. coli to obtain a cell precipitate; and
(d) purifying a peptide having deacetylase activity or a peptide having sulfotransferase activity from the cell precipitate.

In addition, the present disclosure provides a pharmaceutical composition for preventing or treating a neurological disease, including a peptide having deacetylase activity or a peptide having sulfotransferase activity as an active ingredient.

As used herein, the term “neurological disease” is intended to include a disease of the nervous system and a degenerative brain disease.

As used herein, the term “treatment” refers to any action of ameliorating or beneficially altering the symptoms of a neurological disorder by administering the composition of the present disclosure.

As used herein, the term “prevention” refers to any action by which the likelihood of developing a neurological disease or condition is inhibited or delayed by administering the composition of the present disclosure.

The pharmaceutical composition may include a pharmaceutically acceptable carrier. The term “pharmaceutically acceptable carrier” may mean a carrier or diluent that does not interfere with the biological activities and properties of the compound being administered, and does not irritate the living organism. Here, the term “pharmaceutically acceptable” means does not inhibit the activity of the active ingredient and does not have a toxicity greater than the subject to whom it is applied (prescribed) may tolerate.

The type of the carrier used in the present disclosure may be any carrier commonly used and pharmaceutically acceptable in the art. Non-limiting examples of the carriers include saline, sterile water, Ringer's solution, buffered saline, albumin injection solution, dextrose solution, maltodextrin solution, glycerol, ethanol, and the like. These may be used alone or in combinations of two or more. The pharmaceutical composition may be prepared as an oral formulation or a parenteral formulation, including a pharmaceutically acceptable carrier in addition to the active ingredient by any conventional method known in the art, depending on the route of administration.

The pharmaceutical composition can be formulated and used in the form of oral formulations such as powders, granules, tablets, capsules, suspensions, emulsions, syrups, aerosols; external preparations; suppositories; or sterile injectable solutions, respectively, according to the conventional methods. The pharmaceutical composition may be prepared by adding diluents or excipients such as commonly used fillers, extenders, binders, wetting agents, disintegrating agents, or surfactants.

When the pharmaceutical composition is prepared as an oral formulation, it may be prepared in the form of powders, granules, tablets, pills, dragees, capsules, liquids, gels, syrups, suspensions, wafers, etc., in combination with suitable carriers according to the methods known in the art. Examples of suitable pharmaceutically acceptable carriers include sugars such as lactose, glucose, sucrose, dextrose, sorbitol, mannitol, and xylitol; starches such as corn starch, potato starch, and wheat starch; celluloses such as cellulose, methylcellulose, ethylcellulose, sodium carboxymethylcellulose, and hydroxypropylmethylcellulose; polyvinyl pyrrolidone, water, methylhydroxybenzoate, propylhydroxybenzoate, magnesium stearate, mineral oil, malt, gelatin, talc, polyols, and vegetable oils. The pharmaceutical composition may be formulated with diluents and/or excipients such as fillers, extenders, binders, wetting agents, disintegrants, and surfactants, as needed.

When the pharmaceutical composition is prepared as a parenteral formulation, it may be formulated in the form of injections, transdermal agents, nasal inhalers, and suppositories together with suitable carriers according to the methods known in the art. For injectable formulations, suitable carriers include sterile water, ethanol, polyols such as glycerol or propylene glycol, or mixtures thereof, and preferably, Ringer's solution, phosphate buffered saline (PBS) containing triethanolamine, sterile water for injection, or an isotonic solution such as 5% dextrose may be used. When the pharmaceutical composition is formulated for transdermal agents, it may be formulated in the form of ointment, cream, lotion, gel, external solution, paste, liniment, aerosol, etc. For nasal inhalers, they can be formulated in the form of an aerosol spray using a suitable propellant such as dichlorofluoromethane, trichlorofluoromethane, dichlorotetrafluoroethane, or carbon dioxide, and for suppositories, the bases that may be used include witepsol, Tween 61, polyethylene glycols, cacao butter, laurin butter, polyoxyethylene sorbitan fatty acid esters, polyoxyethylene stearates, and sorbitan fatty acid esters.

The pharmaceutical composition may be administered in a “pharmaceutically effective amount” which means an amount sufficient to treat or prevent a disease at a reasonable benefit/risk ratio applicable to medical treatment or prevention, and the effective dose level may be determined depending on the severity of the disease, the activity of the drug, the patient's age, weight, health, and sex, the patient's sensitivity to the drug, the time of administration, the route of administration and the excretion rate of the composition of the present disclosure used, the duration of the treatment, the drugs used in combination with or concurrently with the composition of the present disclosure used, and other factors well known in the medical field. The pharmaceutical composition may be administered alone or in combination with components known to exhibit therapeutic effects against known cancer diseases. It is important to administer the minimum dose that will achieve the maximum effect without side effects, taking all the above-mentioned factors into account.

The dosage of the pharmaceutical composition may be determined by those skilled in the art, taking into account the purpose of use, the severity of the disease, the patient's age, weight, sex, and medical history, or the type of substance used as an active ingredient. For example, the pharmaceutical composition of the present disclosure may be administered at an amount of from about 0.1 ng to about 1,000 mg/kg per adult, preferably 1 ng to about 100 mg/kg, and the frequency of administration of the composition of the present disclosure is not particularly limited, but may be administered once a day or administered several times a day in divided doses. The dosage or frequency of administration is not intended to limit the scope of the present disclosure in any way.

The present disclosure also provides a method for preventing or treating a neurological disease including administering to a subject the peptide of the present disclosure.

As used herein, “subject” includes, without limitation, mammals including mice, livestock, humans, and the like, birds, reptiles, farmed fish, etc., that are suffering from or at risk of suffering from a neurological disease.

The pharmaceutical composition may be administered in a single dose or multiple doses in a pharmaceutically effective amount. In this case, the composition may be formulated and administered in the form of liquid, powder, aerosol, injection, infusion (Ringel), capsule, pill, tablet, suppository, or patch. The route of administration of the pharmaceutical composition for preventing or treating cancer may be via any conventional route as long as it may reach the target tissue.

The pharmaceutical composition may be administered via a route such as intraperitoneal administration, intravenous administration, intramuscular administration, subcutaneous administration, intradermal administration, transdermal patch administration, oral administration, intranasal administration, intrapulmonary administration, or rectal administration, depending on the intended purpose, but is not particularly limited thereto.

The specific information for NDST3 (TO) or NDST4 (T4) and its variants (T1 to T3 and T5 to T7) described herein is shown in Table 1 below.

TABLE 1

| | |
|---|---|
| T0 = Human N-Deacetylase And N-Sulfotransferase 3 (1-873 a.a) | |
| MSFIMKLHRHFQRTVILLATFCMVSIIISAYYLYSGYKQENELSETASEVDCGDLQHLPYQLMEV | SEQ |
| KAMKLFDASRTDPTVLVFVESQYSSLGQDIIMILESSRFQYHIEIAPGKGDLPVLIDKMKGKYILII | ID |
| YENILKYINMDSWNRSLLDKYCVEYGVGVIGFHKTSEKSVQSFQLKGFPFSIYGNLAVKDCCINP | NO |
| HSPLIRVTKSSKLEKGSLPGTDWTVFQINHSAYQPVIFAKVKTPENLSPSISKGAFYATIIHDLGLH | 1 |
| DGIQRVLFGNNLNFWLHKLIFIDAISFLSGKRLTLSLDRYILVDIDDIFVGKEGTRMNTNDVKALL | |
| DTQNLLRAQITNFTFNLGFSGKFYHTGTEEEDEGDDCLLGSVDEFWWFPHMWSHMQPHLFHNE | |
| SSLVEQMILNKKFALEHGIPTDMGYAVAPHHSGVYPVHVQLYEAWKKVWNIKITSTEEYPHLKPA | |
| RYRRGFIHKNIMVLPRQTCGLFTHTIFYKEYPGGPKELDKSIQGGELFFTVVLNPISIFMTHLSNY | |
| GNDRLGLYTFVNLANFVKSWTNLRLQTLPPVQLAHKYFELFPDQKDPLWQNPCDDKRHRDIWS | |
| KEKTCDRLPKFLVIGPQKTGTTALYLFLVMHPSILSNSPSPKTFEEVQFFNRNNYHRGIDWYMDF | |
| FPVPSNVTTDFLFEKSANYFHSEEAPKRAASLVPKAKIITILIDPSDRAYSWYQHQRSHEDPAALK | |
| FSFYEVISAGPRAPSELRALQKRCLVPGWYASHIERWLVYFPPFQLLIIDGQQLRTDPATVMDEVQ | |
| KFLGVLPHYNYSEALTFDSHKGFWCQLLEEGKTKCLGKSKGRKYPPMDSDSRTFLSSYYRDHN | |
| VELSKLLHKLGQPLPSWLRQELQKVR | |
| T1 = ΔTM_Human N-Deacetylase And N-Sulfotransferase 3 (72-873 a.a) | |
| DASRTDPTVLVFVESQYSSLGQDIIMILESSRFQYHIEIAPGKGDLPVLIDKMKGKYILIIYENILKY | SEQ |
| INMDSWNRSLLDKYCVEYGVGVIGFHKTSEKSVQSFQLKGFPFSIYGNLAVKDCCINPHSPLIRV | ID |
| TKSSKLEKGSLPGTDWTVFQINHSAYQPVIFAKVKTPENLSPSISKGAFYATIIHDLGLHDGIQRVL | NO |
| FGNNLNFWLHKLIFIDAISFLSGKRLTLSLDRYILVDIDDIFVGKEGTRMNTNDVKALLDTQNLLR | 2 |
| AQITNFTFNLGFSGKFYHTGTEEEDEGDDCLLGSVDEFWWFPHMWSHMQPHLFHNESSLVEQM | |
| ILNKKFALEHGIPTDMGYAVAPHHSGVYPVHVQLYEAWKKVWNIKITSTEEYPHLKPARYRRGFI | |
| HKNIMVLPRQTCGLFTHTIFYKEYPGGPKELDKSIQGGELFFTVVLNPISIFMTHLSNYGNDRLGL | |
| YTFVNLANFVKSWTNLRLQTLPPVQLAHKYFELFPDQKDPLWQNPCDDKRHRDIWSKEKTCDR | |
| LPKFLVIGPQKTGTTALYLFLVMHPSILSNSPSPKTFEEVQFFNRNNYHRGIDWYMDFFPVPSNVT | |
| TDFLFEKSANYFHSEEAPKRAASLVPKAKIITILIDPSDRAYSWYQHQRSHEDPAALKFSFYEVISA | |
| GPRAPSELRALQKRCLVPGWYASHIERWLVYFPPFQLLIIDGQQLRTDPATVMDEVQKFLGVLPH | |
| YNYSEALTFDSHKGFWCQLLEEGKTKCLGKSKGRKYPPMDSDSRTFLSSYYRDHNVELSKLLH | |
| KLGQPLPSWLRQELQKVR | |
| T2 = Human NDST3_N-Deacetylase domain (72-569 a.a) | |
| DASRTDPTVLVFVESQYSSLGQDIIMILESSRFQYHIEIAPGKGDLPVLIDKMKGKYILIIYENILKY | SEQ |
| INMDSWNRSLLDKYCVEYGVGVIGFHKTSEKSVQSFQLKGFPFSIYGNLAVKDCCINPHSPLIRV | ID |
| TKSSKLEKGSLPGTDWTVFQINHSAYQPVIFAKVKTPENLSPSISKGAFYATIIHDLGLHDGIQRVL | NO |
| FGNNLNFWLHKLIFIDAISFLSGKRLTLSLDRYILVDIDDIFVGKEGTRMNTNDVKALLDTQNLLR | 3 |
| AQITNFTFNLGFSGKFYHTGTEEEDEGDDCLLGSVDEFWWFPHMWSHMQPHLFHNESSLVEQM | |
| ILNKKFALEHGIPTDMGYAVAPHHSGVYPVHVQLYEAWKKVWNIKITSTEEYPHLKPARYRRGFI | |
| HKNIMVLPRQTCGLFTHTIFYKEYPGGPKELDKSIQGGELFFTVVLNPISIFMTHLSNYGNDRLGL | |
| YTFVNLANFVKSWTNLRLQTLPPVQLAHKYFELFPDQK | |
| T3 = Human NDST3_N-Sulfotransferase domain (569-873 a.a) | |
| KDPLWQNPCDDKRHRDIWSKEKTCDRLPKFLVIGPQKTGTTALYLFLVMHPSILSNSPSPKTFEEV | SEQ |
| QFFNRNNYHRGIDWYMDFFPVPSNVTTDFLFEKSANYFHSEEAPKRAASLVPKAKIITILIDPSDR | ID |
| AYSWYQHQRSHEDPAALKFSFYEVISAGPRAPSELRALQKRCLVPGWYASHIERWLVYFPPFQLL | NO |
| IIDGQQLRTDPATVMDEVQKFLGVLPHYNYSEALTFDSHKGFWCQLLEEGKTKCLGKSKGRKYP | 4 |
| PMDSDSRTFLSSYYRDHNVELSKLLHKLGQPLPSWLRQELQKVR | |
| T4 = Human N-Deacetylase And N-Sulfotransferase 4 (1-873 a.a) | |
| MNLIVKLRRSFRTLIVLLATFCLVSIVISAYFLYSGYKQEMTLIETTAEAECTDIKILPYRSMELKTV | SEQ |
| KPIDTSKTDPTVLLFVESQYSQLGQDIIAILESSRFQYHMVIAPGKGDIPPLTDNGKGKYTLVIYEN | ID |
| ILKYVSMDSWNRELLEKYCVEYSVSIIGFHKANENSLPSTQLKGFPLNLFNNLALKDCFVNPQSP | NO |
| LLHITKAPKVEKGPLPGEDWTIFQYNHSTYQPVLLTELQTEKSLSSLSSKTLFATVIQDLGLHDGI | 5 |
| QRVLFGNNLNFWLHKLIFIDAISFLSGKRLTLSLDRYILVDIDDIFVGKEGTRMNVKDVKALLETQ | |
| NLLRTQVANFTFNLGFSGKFYHTGTEEEDEGDDLLLRSVDEFWWFPHMWSHMQPHLFHNESSL | |
| VEQMILNKEFALEHGIPINMGYAVAPHHSGVYPVHIQLYAAWKKVWGIQVTSTEEYPHLKPARYR | |
| KGFIHNSIMVLPRQTCGLFTHTIFYKEYPGGPQELDKSIRGGELFLTILLNPISIFMTHLSNYGNDR | |
| LGLYTFVNLVNFVQSWTNLKLQTLPPVQLAHQYFELFPEQKDPLWQNPCDDKRHKDIWSREKT | |
| CDHLPKFLVIGPQKTGTTALYLFLLMHPSIISNLPSPKTFEEVQFFNGNNYHKGIDWYMDFFPTPS | |
| NTTSDFLFEKSANYFHSEEAPRRAASLVPKAKIITILIDPSDRAYSWYQHQRSHEDPAALRFNFYE | |
| VISTGHWAPSDLKTLQRRCLVPGWYAVHIERWLTYFATSQLLIIDGQQLRSDPATVMDEVQKFLG | |
| VTPRYNYSEALTFDPQKGFWCQLLEGGKTKCLGKSKGRKYPPMDPESRTFLSNYYRDHNVELS | |
| KLLHRLGQPLPSWLRQELQKVR | |
| T5 = ATM_Human N-Deacetylase And N-Sulfotransferase 4 (72-873 a.a) | |
| DTSKTDPTVLLFVESQYSQLGQDIIAILESSRFQYHMVIAPGKGDIPPLTDNGKGKYTLVIYENILK | SEQ |
| YVSMDSWNRELLEKYCVEYSVSIIGFHKANENSLPSTQLKGFPLNLFNNLALKDCFVNPQSPLLH | ID |
| ITKAPKVEKGPLPGEDWTIFQYNHSTYQPVLLTELQTEKSLSSLSSKTLFATVIQDLGLHDGIQRV | NO |
| LFGNNLNFWLHKLIFIDAISFLSGKRLTLSLDRYILVDIDDIFVGKEGTRMNVKDVKALLETQNLL | 6 |
| RTQVANFTFNLGFSGKFYHTGTEEEDEGDDLLLRSVDEFWWFPHMWSHMQPHLFHNESSLVEQ | |
| MILNKEFALEHGIPINMGYAVAPHHSGVYPVHIQLYAAWKKVWGIQVTSTEEYPHLKPARYRKGF | |
| IHNSIMVLPRQTCGLFTHTIFYKEYPGGPQELDKSIRGGELFLTILLNPISIFMTHLSNYGNDRLGL | |
| YTFVNLVNFVQSWTNLKLQTLPPVQLAHQYFELFPEQKDPLWQNPCDDKRHKDIWSREKTCDH | |
| LPKFLVIGPQKTGTTALYLFLLMHPSIISNLPSPKTFEEVQFFNGNNYHKGIDWYMDFFPTPSNTTS | |
| DFLFEKSANYFHSEEAPRRAASLVPKAKIITILIDPSDRAYSWYQHQRSHEDPAALRFNFYEVISTG | |
| HWAPSDLKTLQRRCLVPGWYAVHIERWLTYFATSQLLIIDGQQLRSDPATVMDEVQKFLGVTPRY | |
| NYSEALTFDPQKGFWCQLLEGGKTKCLGKSKGRKYPPMDPESRTFLSNYYRDHNVELSKLLHR | |
| LGQPLPSWLRQELQKVR | |
| T6 = Human NDST4_N-Deacetylase domain (72-569 a.a) | |
| DTSKTDPTVLLFVESQYSQLGQDIIAILESSRFQYHMVIAPGKGDIPPLTDNGKGKYTLVIYENILK | SEQ |
| YVSMDSWNRELLEKYCVEYSVSIIGFHKANENSLPSTQLKGFPLNLFNNLALKDCFVNPQSPLLH | ID |
| ITKAPKVEKGPLPGEDWTIFQYNHSTYQPVLLTELQTEKSLSSLSSKTLFATVIQDLGLHDGIQRV | NO |
| LFGNNLNFWLHKLIFIDAISFLSGKRLTLSLDRYILVDIDDIFVGKEGTRMNVKDVKALLETQNLL | 7 |
| RTQVANFTFNLGFSGKFYHTGTEEEDEGDDLLLRSVDEFWWFPHMWSHMQPHLFHNESSLVEQ | |
| MILNKEFALEHGIPINMGYAVAPHHSGVYPVHIQLYAAWKKVWGIQVTSTEEYPHLKPARYRKGF | |
| IHNSIMVLPRQTCGLFTHTIFYKEYPGGPQELDKSIRGGELFLTILLNPISIFMTHLSNYGNDRLGL | |
| YTFVNLVNFVQSWTNLKLQTLPPVQLAHQYFELFPEQK | |
| T7 = Human NDST4_N-Sulfotransferase domain (569-873 a.a) | |
| KDPLWQNPCDDKRHKDIWSREKTCDHLPKFLVIGPQKTGTTALYLFLLMHPSIISNLPSPKTFEEV | SEQ |
| QFFNGNNYHKGIDWYMDFFPTPSNTTSDFLFEKSANYFHSEEAPRRAASLVPKAKIITILIDPSDR | ID |
| AYSWYQHQRSHEDPAALRFNFYEVISTGHWAPSDLKTLQRRCLVPGWYAVHIERWLTYFATSQL | NO |
| LIIDGQQLRSDPATVMDEVQKFLGVTPRYNYSEALTFDPQKGFWCQLLEGGKTKCLGKSKGRKY | 8 |
| PPMDPESRTFLSNYYRDHNVELSKLLHRLGQPLPSWLRQELQKVR | |

Additional aspects of embodiments will be set forth in part in the description which follows and, in part, will be apparent from the description, or may be learned by practice of the disclosure.

The present disclosure identifies a domain having N-deacetylase or N-sulfotransferase activity in the NDST3 and NDST4 proteins, and confirms that using vectors containing NDST3 or NDST4 derived peptide genes including said domains, the peptides may be mass-produced in a soluble form in E. coli. Therefore, it is expected that the peptide of the present disclosure will be used as a platform for developing protein drugs or inhibitors thereof utilizing NDST3 and NDST4 proteins.

### BRIEF DESCRIPTION OF THE DRAWINGS

The above and other aspects, features, and advantages of certain embodiments of the disclosure will be more apparent from the following description taken in conjunction with the accompanying drawings.

FIG. 1A is a schematic diagram showing the functional domains of the human NDST3 protein.

FIG. 1B is a schematic diagram showing the functional domains of the human NDST4 protein.

FIG. 2A is a schematic diagram of a human derived NDST3-derived protein expression DNA vector.

FIG. 2B is a schematic diagram of a human derived NDST4-derived protein expression DNA vector.

FIGS. 3A, 3B, 3C, and 3D show the results of identifying the production of a water-soluble protein designed in the present disclosure. E. coli transformed with expression vectors encoding each human DNST3 domain was cultured and lysed, and then Western blot was performed to determine the level of the protein expression and yield. ‘M’ represents a protein marker (kDa), ‘I−’ represents induction of protein expression, ‘I’ represents cell lysates after protein expression induction, ‘S’ represents the supernatant obtained by centrifugation after cell lysis, and ‘P’ represents the status of the expressed insoluble protein.

FIGS. 4A, 4B, 4C, and 4D show the results of identifying the production of a water-soluble protein designed in the present disclosure. E. coli transformed with expression vectors encoding each human DNST4 domain was cultured and lysed, and then Western blot was performed to determine the level of the protein expression and yield. ‘M’ represents a protein marker (kDa), ‘I−’ represents induction of protein expression, ‘I’ represents cell lysates after protein expression induction, ‘S’ represents the supernatant obtained by centrifugation after cell lysis, and ‘P’ represents the status of the expressed insoluble protein.

FIGS. 5A, 5B, and 5C show the results of culturing and lysing E. coli transformed with an expression vector encoding each domain of human DNST3, and then performing affinity chromatography and size exclusion chromatography using a nickel (Ni-NTA) column to separate and purify the water-soluble protein designed in the present disclosure. ‘C’ represents the cell lysate after protein expression induction, ‘S’ represents the supernatant obtained by centrifugation after cell lysis, ‘FT’ represents the column flow-through, ‘4%’ represents the column flow-through after a 20 mM imidazole wash, ‘10%’ is the column flow-through after a 50 mM imidazole wash, and ‘before cut’ and ‘after cut’ indicate before and after cleavage of the eight histidine tags using PreScission protease, respectively. ‘E’ or elution represents the fraction following an imidazole concentration gradient or each fraction separated and purified by protein exclusion chromatography.

FIGS. 6A and 6B show the results of isolating and purifying a water-soluble protein designed in the present disclosure by culturing and lysing E. coli transformed with an expression vector encoding each domain of human DNST4, and then performing affinity chromatography and size exclusion chromatography using a nickel (Ni-NTA) column. ‘C’ represents the cell lysate after protein expression induction, ‘S’ represents the supernatant obtained by centrifugation after cell lysis, ‘FT’ represents the column flow-through, ‘4%’ represents the column flow-through after a 20 mM imidazole wash, ‘10%’ is the column flow-through after a 50 mM imidazole wash, and ‘before cut’ and ‘after cut’ indicate before and after cleavage of the eight histidine tags using PreScission protease, respectively. ‘E’ or elution represents the fraction following an imidazole concentration gradient or each fraction separated and purified by protein exclusion chromatography.

FIG. 7 shows the results of evaluating the enzyme activity of proteins derived from human NDST3 and NDST4 in vitro.

### DETAILED DESCRIPTION

Hereinafter, specific embodiments will be described in detail with reference to the accompanying drawings, although the present disclosure may have various embodiments and various changes may be made to the embodiments. However, such illustrations and descriptions are not intended to limit the present disclosure to specific embodiments, and should be understood as including all transformations, equivalents, and substitutes that may be included in the spirit and scope of the present disclosure. In the description of the present disclosure, any detailed description of well-known related structures or functions has been omitted where it is deemed that such description will cause ambiguous interpretation of the present disclosure.

### EXAMPLE

### Example 1. Amplification and Expression of Human NDST3 and NDST4 Variant Genes

### 1-1. Amplification of Human NDST3 and NDST4 Variant Genes

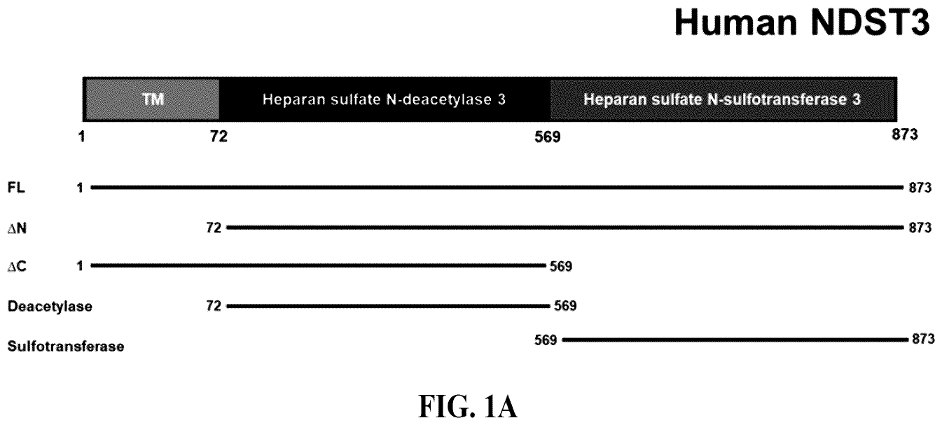

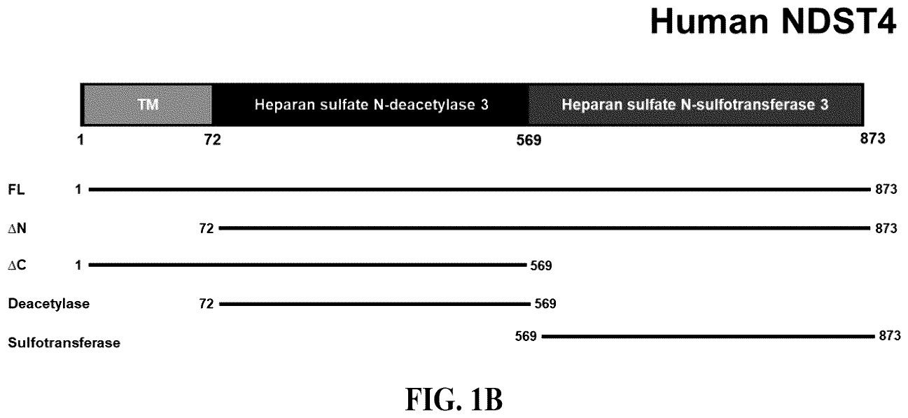

A human NDST3 variant protein having an amino acid sequence from aspartic acid (D) terminus at position 72 (N-terminus) to arginine (R) terminus at position 873, identified by SEQ ID NO: 2; an amino acid sequence from aspartic acid (D) terminus at position 72 (N-terminus) to lysine (K) terminus at position 569, identified by SEQ ID NO: 3; and an amino acid sequence from lysine (K) terminus at position 569 to arginine (R) at position 873, identified by SEQ ID NO. 4, and a human NDST4 variant protein having an amino acid sequence from aspartic acid (D) terminus at position 72 to arginine (R) terminus at position 873, identified by SEQ ID NO. 6; an amino acid sequence from aspartic acid (D) terminus at position 72 (N-terminus) to lysine (K) terminus at position 569, identified by SEQ ID NO. 7; and an amino acid sequence from lysine (K) terminus at position 569 (N-terminus) to arginine (R) terminus at position 873, identified by SEQ ID NO. 8 were used as coding sequences. Also, to facilitate purification of the protein to be produced, a histidine fusion (His-tag) protein, glutathione S-transferase (GST), or thioredoxin (trx) may be conjugated to the N-terminus. Each sequence is derived from human NDST3 or NDST4 proteins, and each domain of the proteins is indicated in FIGS. 1A and 1B. For the synthesis and amplification of a gene, a PCR reaction was performed using the following primers synthesized using a nucleic acid synthesizer. Each PCR reaction was performed with a gene amplifier using synthesized primers (Table 2) for amplification of each gene fragment.

TABLE 2

| | | | SEQ |
|---|---|---|---|
| Template DNA | Direction | Primer Sequence (5′→3′) | ID NO. |
| HUMAN NDST3 full | Forward | 5′-caccggatccatgagttttatcatgaagcttc-3′ | 9 |
| (1-873 a.a) | Reverse | 5′-gctaggcatctcgagttactatcttactttctgcagctc-3′ | 10 |
| HUMAN NDST3 ΔTM | Forward | 5′-caccggatccatggatgcctcaaggacagac-3′ | 11 |
| (72-873 a.a) | Reverse | 5′-gctaggcatctcgagttactatcttactttctgcagctc-3′ | 12 |
| HUMAN NDST3 N- | Forward | 5′-caccggatccatgagttttatcatgaagcttc-3′ | 13 |
| Deacetylase | Reverse | 5′gctaggcatctcgagttactatttctgatcaggaaacagc-3′ | 14 |
| (1-569 a.a) | | | |
| HUMAN NDST3 ΔTM N- | Forward | 5′-caccggatccatggatgcctcaaggacagac-3′ | 15 |
| Deacetylase | Reverse | 5′-gctaggcatctcgagttaCTAtttctgatcaggaaacagc-3′ | 16 |
| (72-569 a.a) | | | |
| HUMAN NDST3 N- | Forward | 5′-caccggatccatggaccctctctggcagaatcc-3′ | 17 |
| Sulfotransferase | Reverse | 5′-gctaggcatctcgagttactatcttactttctgcagctc-3′ | 18 |
| (569-873 a.a) | | | |
| HUMAN NDST4 Full | Forward | 5′-caccggatccatgaatcttattgtgaa-3′ | 19 |
| (1-873 a.a) | Reverse | 5′-gctaggcatctcgagttatctcactttctgcagttcc-3′ | 20 |
| HUMAN NDST4 ΔTM | Forward | 5′-caccggatccatggacacatccaaaacgg-3′ | 21 |
| (72-873 a.a) | Reverse | 5′-gctaggcatctcgagttatctcactttctgcagttcc-3′ | 22 |
| HUMAN NDST4 N- | Forward | 5′-caccggatccatgaatcttattgtgaa-3′ | 23 |
| Deacetylase | Reverse | 5′-gctaggcatctcgagttagtctttctgctcagggaag-3′ | 24 |
| (1-569 a.a) | | | |
| HUMAN NDST4 ΔTM N- | Forward | 5′-caccggatccatggatgcctcaaggacagac-3′ | 25 |
| Deacetylase | Reverse | 5′-gctaggcatctcgagttagtctttctgctcagggaag-3′ | 26 |
| (72-569 a.a) | | | |
| HUMAN NDST4 N- | Forward | 5′-caccggatccatggaccctctatggcagaa | 27 |
| Sulfotransferase | Reverse | 5′-gctaggcatctcgagttatctcactttctgcagttcc-3′ | 28 |
| (569-873 a.a) | | | |

The forward primer includes a sequence corresponding to the BamH1 restriction enzyme recognition site (SEQ ID NOs: 9, 11, 13, 14, 16, 18), and the reverse primer includes a sequence corresponding to the XhoI restriction enzyme recognition site (SEQ ID NOs: 10, 12, 15, 17).

Using human NDST3 and human NDST4 as templates, PCR was performed as follows: A reaction solution was prepared by adding 79 μl of distilled water to a mixed solution of 1 μl of the human NDST3 and human NDST4 full-length cDNA, 8 μl of 2.5 mMv dNTP, 1 μl of each 100 pmol forward primer (SEQ ID NOs: 9, 11, 13, 14, 16, 18) and reverse primer (SEQ ID NOs: 10, 12, 15, 17), 1 μl of PfuTaq DNA polymerase (5 U/μl, Stratagene, USA), and 10 μl of PCR buffer solution (Stratagene), and the reaction solution was reacted at 82° C. for 2 minutes, at 94° C. for 1 minute, at 94° C. for 30 seconds, a 56° C. for 1 minute 30 seconds, and at 72° C. for 4 minutes 30 seconds. This reaction was repeated 30 times. The reaction solution was separated by electrophoresis on a 0.8% agarose gel to elute the genes. To 16 μl of the obtained eluate, 2 μl of a 10-fold restriction enzyme reaction buffer solution and 1 μl each of restriction enzymes BamH1 (New England Biolabs (NEB), USA) and Xho I (NEB, USA) were added, and the mixture was reacted at 37° C. for 16 hours. The obtained reaction solution was separated by electrophoresis on a 0.8% agarose gel to extract DNA fragments coding for the desired human NDST3 and NDST4 protein variants, and these were dissolved in 50 μl of distilled water solution. Each was designated as HUMAN_NDST3 ΔTM (hNDST3NΔTM) B/X, HUMAN NDST3_N-Deacetylase (hNDST3DA) B/X, HUMAN_NDST3 ΔTM N-Deacetylase (hNDST3ΔTMDA) B/X, HUMAN NDST3 N-Sulfotransferase (hNDST3ST) B/X, HUMAN_NDST4 ΔTM (hNDST4NΔTM) B/X, HUMAN NDST4_N-Deacetylase (hNDST4DA) B/X, HUMAN_NDST4 ΔTM N-Deacetylase (hNDST4ΔTMDA) B/X and HUMAN NDST4 N-Sulfotransferase (hNDST4ST) B/X.

### 1-2. Preparation of Human NDST3 and NDST4 Variant Expression Vectors

Plasmid pET-32a (Novagene Inc., USA) expresses thioredoxin and six histidines at the N-terminus, pGEX4T-1 (Novagene Inc., USA) expresses glutathione S-transferase at the N-terminus, and pET-28a (Novagene Inc., USA) expresses six histidine residues at the N-terminus. These were treated with restriction enzymes BamHI and XhoI, and then DNA fragments of approximately 920 bp in size were isolated by electrophoresis, which were designated as pET-32aB/X, pGEX4T-1B/X, and pET-28aB/X. 0.5 μg of hNDST3NΔTM B/X, hNDST3DAB/X, hNDST3ΔTMDA B/X, hNDST3ST B/X, hNDST4NΔTM B/X, hNDST4DA B/X, hNDST4ΔTMDA B/X and hNDST4ST B/X were placed in a reaction tube with 0.1 μg of pET-32aB/X, pGEX4T-1B/X and pET-28a B/X. Then, 2 μl of 10-fold ligation reaction solution (20 mM Hepe-HCl, pH 7.8, 100 mM MgCl₂, 100 mM DTT and 10 mM ATP), 10 U of T4 DNA ligase, and distilled water was added to a total volume of 20 μl, and the mixture was reacted at 16° C. for 12 hours. This reaction solution was added to competent E. coli BL21 (DE3) (Novagene Inc., USA) or Rosetta (DE3) (Novagene Inc., USA) cells to transform the competent cells. Then, these were plated on 1) kanamycin medium containing 50 μg/ml of LB (1% bactotryptone, 0.5% yeast extract, and 1% sodium chloride), 2) ampicillin medium containing 100 μg/ml of LB (1% bactotryptone, 0.5% yeast extract, and 1% sodium chloride) to select E. coli transformants. From these transformants, the plasmids were extracted, and recombinant plasmids were confirmed by restriction enzymes and sequencing.

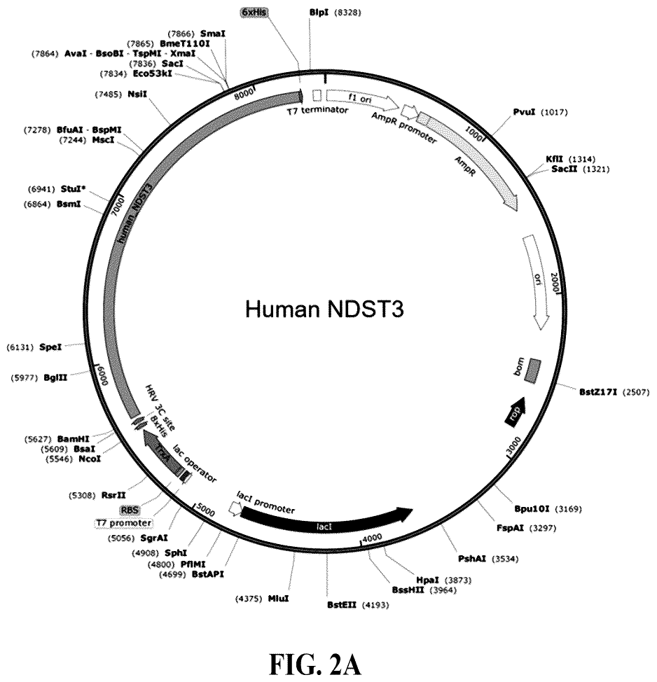

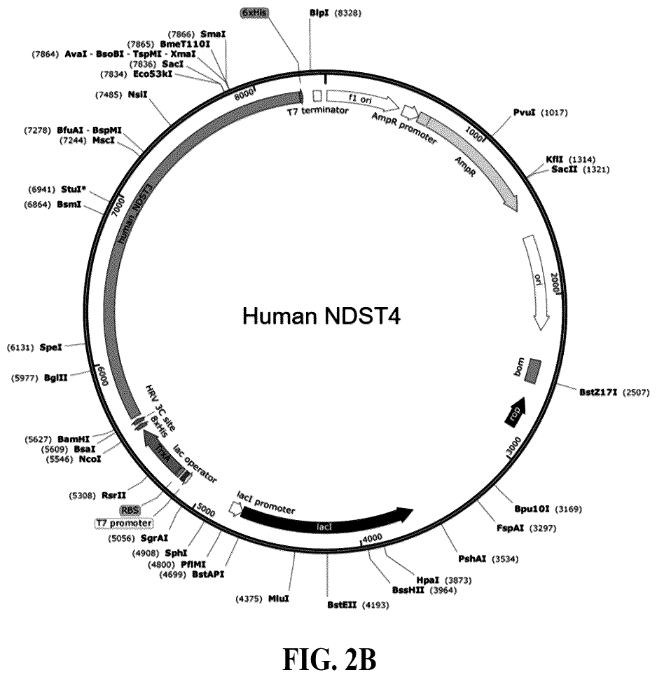

It was identified that hNDST3NΔTM, hNDST3DA, hNDST3ΔTMDA, hNDST3ST, hNDST4NΔTM, hNDST4DA, hNDST4ΔTMDA and hNDST4ST were obtained, which were made by ligating hNDST3NΔTM B/X, hNDST3DA B/X, hNDST3ΔTMDA B/X, hNDST3ST B/X, hNDST4NΔTM B/X, hNDST4DA B/X, hNDST4ΔTMDA B/X and hNDST4ST B/X fragments prepared in Example 1-1 to the plasmids pET-32aB/X, pGEX4T-1B/X and pET-28a B/X. The process of cloning the coding genes of the human NDST3 and NDST4 variant proteins into the E. coli expression vectors pET32a, pGEX4T-1 and pET28a, respectively, is illustrated in FIGS. 2A and 2B.

### 1-3. Expression of NDST3 and NDST4 Variants in E. Coli

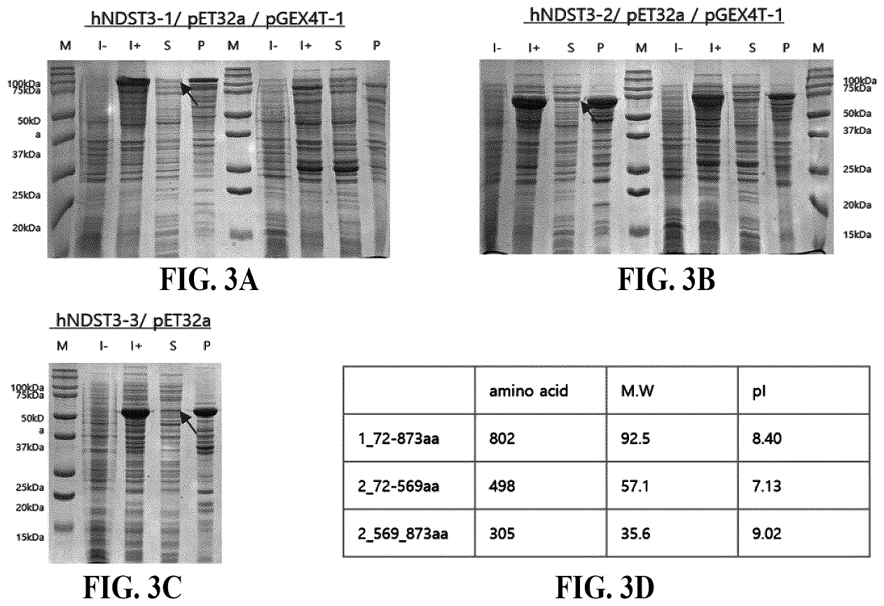

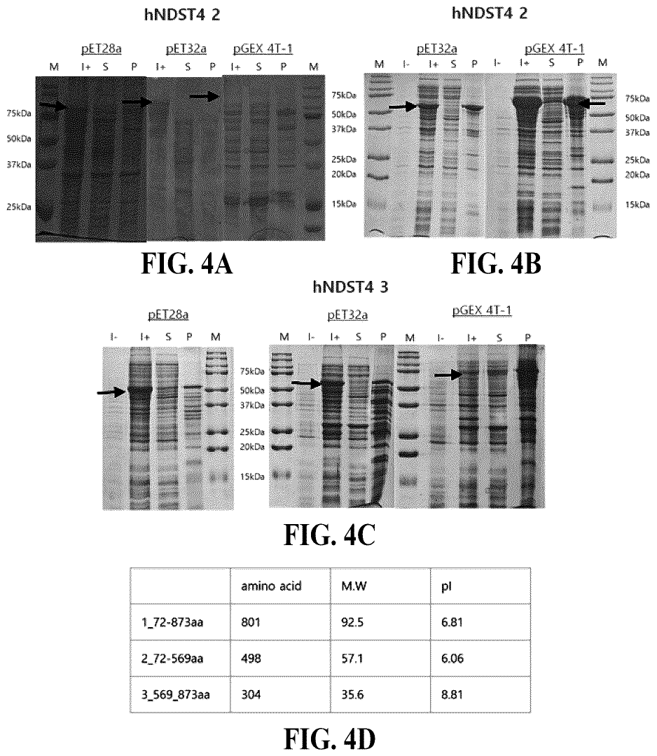

The expression host, E. coli BL21 (DE3) (Novagen Inc., USA) or Rosetta (DE3) (Novagen Inc., USA) was transformed with the expression vectors pET-32a-hNDST3NΔTM, pGEX4T-1-hNDST3NΔTM, pET-28a-hNDST3NΔTM, pET-32a-hNDST3DA, pGEX4T-1-hNDST3DA, pET-28a-hNDST3DA, pET-32a-hNDST3ΔTMDA, pGEX4T-1-hNDST3ΔTMDA, pET-28a-hNDST3ΔTMDA, pET-32a-hNDST3ST, pGEX4T-1-hNDST3ST, pET-28a-hNDST3ST, pET-32a-hNDST4NΔTM, pGEX4T-1-hNDST4NΔTM, pET-28a-hNDST4NΔTM, pET-32a-hNDST4DA, pGEX4T-1-hNDST4DA, pET-28a-hNDST4DA, pET-32a-hNDST4ΔTMDA, pGEX4T-1-hNDST4ΔTMDA, pET-28a-hNDST4ΔTMDA, pET-32a-hNDST4ST, pGEX4T-1-hNDST4ST, pET-28a-hNDST4ST prepared in Example 1-2, by conventional methods. The transformed E. coli strain was cultured by being shaken in LB broth containing 100 μg/mL of kanamycin and 100 μg/mL of ampicillin for 12 hours. Then, 1 mL was taken from the broth and added to 100 mL of LB broth (containing 100 μg/mL of kanamycin and 100 μg/mL of ampicillin). The culture was incubated at 37° C. until the absorbance value of the culture at 600 nm reached approximately 0.6. Then, the culture temperature was lowered to 18° C., and IPTG (isopropyl-ß-D-galactopyranoside) was added at a final concentration of 0.6 mM. 1 mL of each of the cultures was taken before and 16 hours after the IPTG was added, and these were centrifuged at 10,000 g for 2 minutes to collect the respective cell precipitates. The collected precipitates were subjected to 15% SDS-polyacrylamide gel electrophoresis, and then were analyzed by staining the proteins with Coomasie Brilliant Blue (Bio-Rad 161-0400). The results of the SDS-polyacrylamide gel electrophoresis performed on the cell precipitates obtained by culturing E. coli transformed with E. coli expression vectors pET32a, pGEX4T-1, or pET28a, which contain genes coding for the obtained proteins hNDST3NΔTM, hNDST3DA, hNDST3ΔTMDA, hNDST3ST, hNDST4NΔTM, hNDST4DA, hNDST4ΔTMDA and hNDST4ST are shown in FIGS. 3 and 4.

### Example 2. Expression of Recombinant Protein hNDST3NΔTM (72-873Aa) Gene in E. coli

The expression vector pET32a-hNDST3NΔTM (72-873aa) obtained in the above-mentioned step was used to transform the expression host, E. coli BL21 (RIL) by conventional methods. E. coli strains transformed with pET32a-hNDST3NΔTM (72-873aa) were cultured in 5.5 ml of LB broth containing 100 μg/mL of ampicillin at 37° C. for 18 hours. Then, 50 μl of the culture was subcultured in 5.5 ml of LB broth (containing 100 μg/mL of ampicillin). The culture was incubated at 37° C. until the absorbance value of the culture at 600 nm reached approximately 0.2-0.3. Then, the culture temperature was lowered to 18° C., and the culture was incubated until the absorbance value of the culture reached approximately 0.5 to 6. Then, IPTG (isopropyl-ß-D-galactopyranoside) was added thereto at a final concentration of 0.5 mM.

Cells incubated for 18 hours to which IPTG was previously added were centrifuged to collect the respective cell precipitates. The collected precipitates were each suspended in 0.5 ml of cell lysis buffer (300 mM sodium chloride, 20 mM Hepes-HCl buffer, pH 7.5, 5 mM beta-mercaptoethanol), and the cells were disrupted using a mini ultrasonicator (Sonic Dismembrator, Fisher, USA) in ice-cold water. The obtained solution was centrifuged at 13,000 rpm for 10 minutes in a centrifuge to separate the supernatant and the precipitate, which was subjected to 15% SDS-polyacrylamide gel electrophoresis, and analyzed by staining the protein pET32a-hNDST3NΔTM (72-873aa) with Coomassie Brilliant Blue (Bio-Rad 161-0400). The results for each are shown in FIG. 3 (M: protein marker, I−: before IPTG induction, I+: cell lysate sample after IPTG induction, S: supernatant after centrifugation of IPTG-induced sample, P: precipitate after centrifugation of IPTG-induced sample).

### Example 3. Purification of Recombinant Protein hNDST3NΔTM (72-873Aa) in E. coli

3-1. Culturing and Disruption of E. coli Cells

In the way of Example 2, E. coli cells expressing the recombinant protein were cultured in 8 l quantities, these cells were centrifuged at 3,600 rpm for 20 minutes using a centrifuge to collect the E. coli cell precipitate. Then, this precipitate was suspended in the buffer solution containing 400 mM sodium chloride, 20 mM Hepes-HCl buffer, at pH 7.5, and 10 mM beta-mercaptoethanol at 25 ml per liter of cells, and the cells were disrupted using an ultrasonicator (Fisher, USA) in ice-cold water. The obtained solution was centrifuged at 18,000 rpm for 1 hour using a centrifuge to obtain the supernatant.

### 3-2. Purification by Column Chromatography

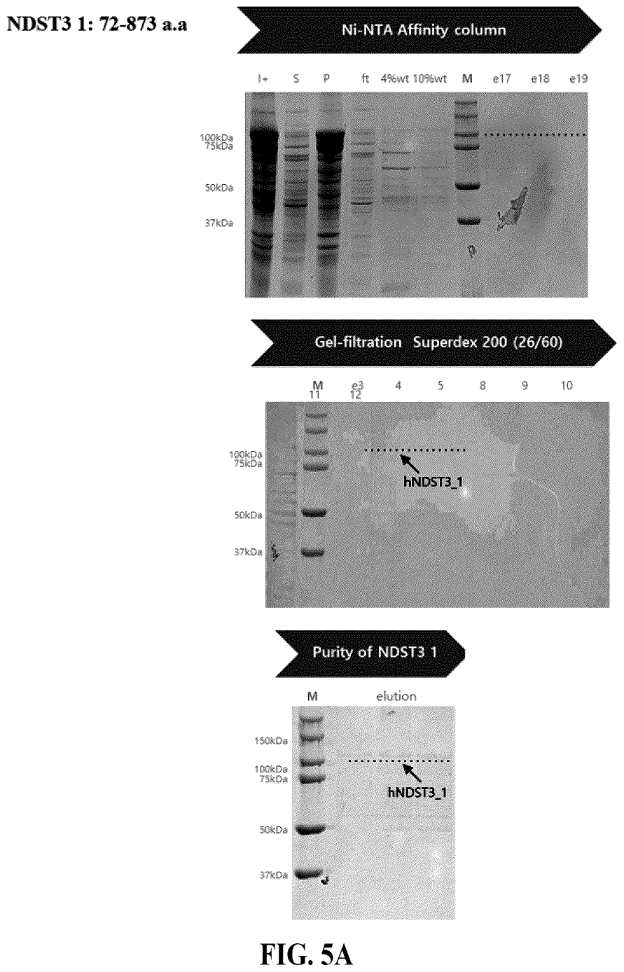

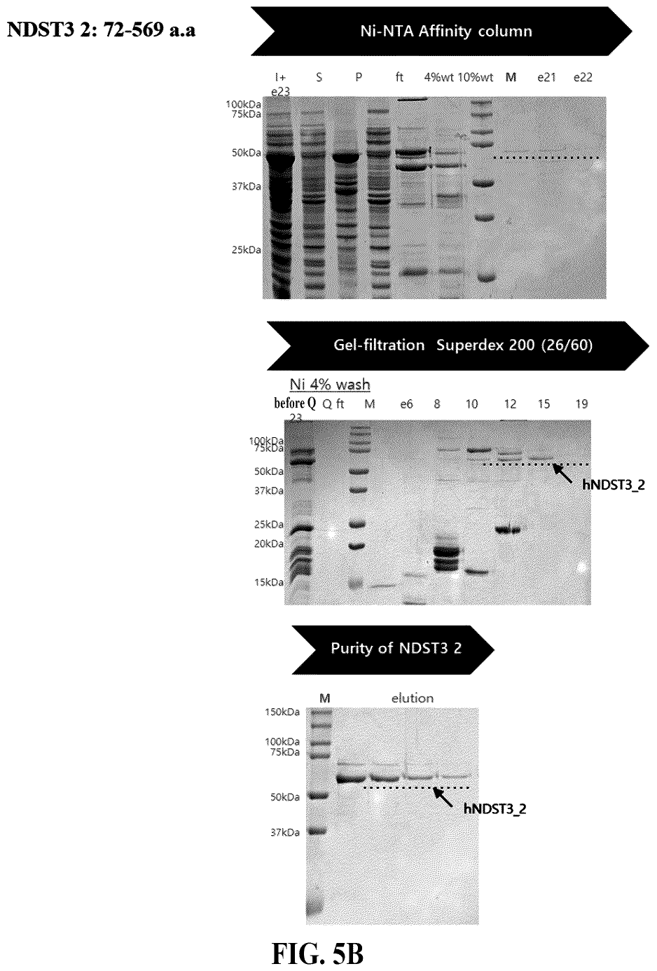

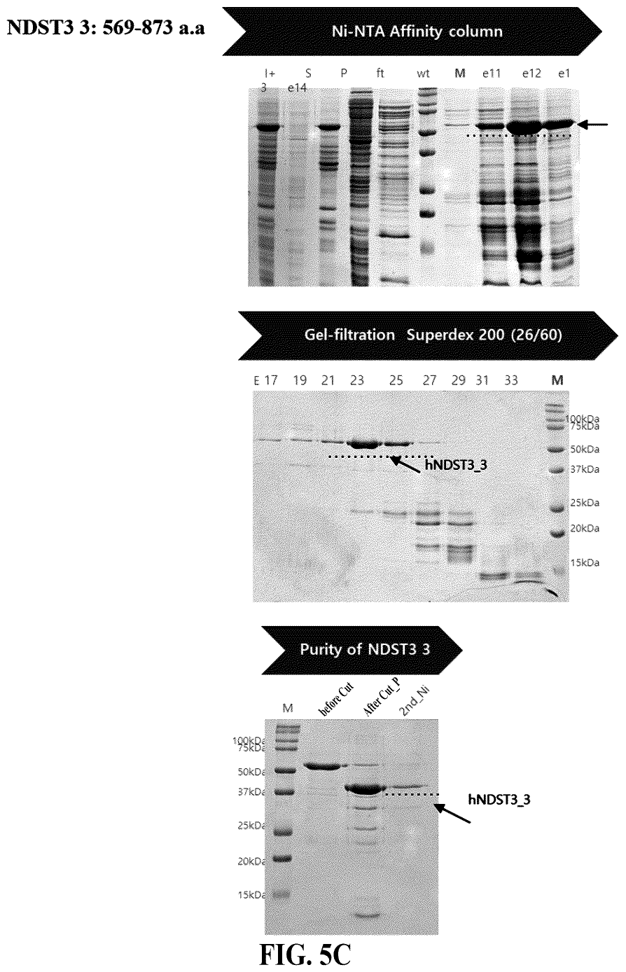

The supernatant of pET32a-hNDST3NΔTM (72-873aa) obtained in Example 3-1 was run on a nickel (Ni-NTA) column (GE Healthcare Life Sciences, USA) pre-equilibrated with the above-mentioned buffer solution, and the proteins were eluted from the column with a 500 mM imidazole concentration gradient using the above-mentioned buffer as the eluent. Each protein-containing fraction was identified by 10% SDS-polyacrylamide gel electrophoresis, and the fractions were pooled. The results for pET32a-hNDST3NΔTM (72-873aa) are shown in FIG. 5A (M: protein marker, I−: before IPTG induction, I+: cell lysate sample after IPTG induction, S: supernatant after centrifugation of IPTG-induced sample, P: precipitate after centrifugation of IPTG-induced sample).

### 3-3. Purification of Proteins by Ion Exchange Chromatography

The volume of the pET32a-hNDST3NΔTM (72-873aa) protein obtained at the step 3-2 of Example 3 was concentrated to about 5 ml, and this volume of protein was injected into a gel permeation column (Superdex 200, 10/300 GL, GE Healthcare Life Sciences, USA) pre-equilibrated with the above-mentioned buffer solution. Then, the proteins were eluted with a buffer solution containing 200 mM sodium chloride, 20 mM Hepes-HCl buffer, at pH 7.5, and 10 mM beta-mercaptoethanol, and these proteins were separated by molecular weight.

SDS-acrylamide gel electrophoresis was performed to collect fractions corresponding to the pET32a-hNDST3NΔTM (72-873aa) protein. The results of the above are shown in FIG. 5A.

### Example 4. Expression of Recombinant Protein hNDST3ΔTMDA Gene in E. coli

The expression vector pET32a-hNDST3ΔTMDA (72-569aa) obtained at the above-mentioned step was used to transform the expression host, E. coli BL21 (RIL) by conventional methods. E. coli strains transformed with pET32a-hNDST3ΔTMDA (72-569aa) were cultured in 5.5 ml of LB broth containing 100 μg/mL of ampicillin at 37° C. for 18 hours. Then, 50 μl of the culture was subcultured in 5.5 ml of LB broth (containing 100 μg/mL of ampicillin). The culture was incubated at 37° C. until the absorbance value of the culture at 600 nm reached approximately 0.2-0.3. Then, the culture temperature was lowered to 18° C., and the culture was incubated until the absorbance value of the culture reached approximately 0.5 to 6. Then, IPTG (isopropyl-ß-D-galactopyranoside) was added thereto at a final concentration of 0.5 mM.

Cells incubated for 18 hours to which IPTG was previously added were centrifuged to collect the respective cell precipitates. The collected precipitates were each suspended in 0.5 ml of cell lysis buffer (300 mM sodium chloride, 20 mM Hepes-HCl buffer, at pH 7.5, 5 mM beta-mercaptoethanol), and the cells were disrupted using a mini ultrasonicator (Sonic Dismembrator, Fisher, USA) in ice-cold water. The obtained solution was centrifuged at 13,000 rpm for 10 minutes in a centrifuge to separate the supernatant and the precipitate, which was subjected to 15% SDS-polyacrylamide gel electrophoresis, and analyzed by staining the protein pET32a-hNDST3 (72-569aa) with Coomassie Brilliant Blue (Bio-Rad 161-0400). The results of the above are shown in FIG. 3 (M: protein marker, I−: before IPTG induction, I+: cell lysate sample after IPTG induction, S: supernatant after centrifugation of IPTG-induced sample, P: precipitate after centrifugation of IPTG-induced sample).

### Example 5. Purification of Recombinant Protein hNDST3ΔTMDA in E. coli

5-1. Culturing and Disruption of E. coli Cells

In the way of Example 2, E. coli cells expressing the recombinant protein were cultured in 8 l quantities, these cells were centrifuged at 3,600 rpm for 20 minutes using a centrifuge to collect the E. coli cell precipitate. Then, this precipitate was suspended in a buffer solution containing 400 mM sodium chloride, 20 mM Hepes-HCl buffer, at pH 7.5, and 10 mM beta-mercaptoethanol at 15 ml per liter of cells, and the cells were disrupted using an ultrasonicator (Fisher, USA) in ice-cold water. The obtained solution was centrifuged at 18,000 rpm for 1 hour using a centrifuge to obtain the supernatant.

### 5-2. Purification by Column Chromatography

The supernatant of pET32a-hNDST3ΔTMDA (72-569aa) obtained in step 1 was run on a nickel (Ni-NTA) column (GE Healthcare Life Sciences, USA) pre-equilibrated with the above-mentioned buffer solution, and the proteins were eluted from the column with a 500 mM imidazole concentration gradient using the above-mentioned buffer above as the eluent. Each protein-containing fraction was identified by 10% SDS-polyacrylamide gel electrophoresis, and the fractions were pooled. The results for pET32a-hNDST3ΔTMDA (72-569aa) are shown in FIG. 5B (M: protein marker, I−: before IPTG induction, I+: cell lysate sample after IPTG induction, S: supernatant after centrifugation of IPTG-induced sample, P: precipitate after centrifugation of IPTG-induced sample).

### 5-3. Purification of Proteins by Ion Exchange Chromatography

The pET32a-hNDST3ΔTMDA (72-569aa) protein obtained in Example 5-2 was diluted more than 3-fold with a buffer solution containing 20 mM Hepes, at pH 7.8, and 10 mM beta-mercaptoethanol, and was run on a Q HP column (GE Healthcare Life Sciences, USA) equilibrated with the above-mentioned buffer solution. Then, the proteins were eluted from the column with a 1 M sodium chloride concentration gradient using the above-mentioned buffer as the eluent.

15% SDS-polyacrylamide gel electrophoresis was performed, and the results for pET32a-hNDST3ΔTMDA (72-569aa) are shown in FIG. 5B.

### Example 6. Expression of the hNDST3ST (569-873Aa) Recombinant Gene in E. coli

The expression vector pET32a-hNDST3ST (569-873aa) obtained at the above-mentioned step was used to transform the expression host, E. coli BL21 (RIL) by conventional methods. E. coli strains transformed with pET32a-hNDST3ST (569-873aa) were cultured in 5.5 ml of LB broth containing 100 μg/mL of ampicillin at 37° C. for 18 hours. Then, 50 μl of the culture was subcultured in 5.5 ml of LB broth (containing 100 μg/mL of ampicillin). The culture was incubated at 37° C. until the absorbance value of the culture at 600 nm reached approximately 0.2-0.3. Then, the culture temperature was lowered to 18° C., and the culture was incubated until the absorbance value of the culture reached approximately 0.5 to 6. Then, IPTG (isopropyl-ß-D-galactopyranoside) was added thereto at a final concentration of 0.5 mM.

Cells incubated for 18 hours to which IPTG was previously added were centrifuged to collect the respective cell precipitates. The collected precipitates were each suspended in 0.5 ml of cell lysis buffer (300 mM sodium chloride, 20 mM Hepes-HCl buffer, at pH 7.5, 5 mM beta-mercaptoethanol), and the cells were disrupted using a mini ultrasonicator (Sonic Dismembrator, Fisher, USA) in ice-cold water. The obtained solution was centrifuged at 13,000 rpm for 10 minutes in a centrifuge to separate the supernatant and the precipitate, which was subjected to 15% SDS-polyacrylamide gel electrophoresis, and analyzed by staining the protein pET32a-hNDST3ST (569-873aa) with Coomassie Brilliant Blue (Bio-Rad 161-0400). The results of the above are shown in FIG. 5C (M: protein marker, I−: before IPTG induction, I+: cell lysate sample after IPTG induction, S: supernatant after centrifugation of IPTG-induced sample, P: precipitate after centrifugation of IPTG-induced sample).

### Example 7. Purification of Recombinant Protein hNDST3ST (569-873Aa) in E. coli

7-1. Culturing and Disruption of E. coli Cells

In the way of Example 2, E. coli cells expressing the recombinant protein were cultured in 4f quantities, these cells were centrifuged at 3,600 rpm for 20 minutes using a centrifuge to collect the E. coli cell precipitate. Then, this precipitate was suspended in a buffer solution containing 400 mM sodium chloride, 20 mM Hepes-HCl buffer, at pH 7.5, and 10 mM beta-mercaptoethanol at 25 ml per liter of cells, and the cells were disrupted using an ultrasonicator (Fisher, USA) in ice-cold water. The obtained solution was centrifuged at 18,000 rpm for 1 hour using a centrifuge to obtain the supernatant.

### 7-2. Purification by Column Chromatography

The supernatant of pET32a-hNDST3ST (569-873aa) obtained in Example 7-1 was run on a nickel (Ni-NTA) column (GE Healthcare Life Sciences, USA) pre-equilibrated with the above-mentioned buffer solution, and the proteins were eluted from the column with a 500 mM imidazole concentration gradient using the above-mentioned buffer as the eluent. Each protein-containing fraction was identified by SDS-polyacrylamide gel electrophoresis, and the fractions were pooled. The results for pET32a-hNDST3ST (569-873aa) are shown in FIG. 5C (M: protein marker, I−: before IPTG induction, I+: cell lysate sample after IPTG induction, S: supernatant after centrifugation of IPTG-induced sample, P: precipitate after centrifugation of IPTG-induced sample).

### 7-3. Separation and Purification of Proteins by Column Chromatography

The volume of the pET32a-hNDST3ST (569-873aa) protein obtained in Example 7-2 was concentrated to about 5 ml, and this volume of protein was injected into a gel permeation column (Superdex 200, 26/60 GL, GE Healthcare Life Sciences, USA) pre-equilibrated with the above-mentioned buffer solution. Then, the proteins were eluted with a buffer solution containing 400 mM sodium chloride, 20 mM Hepes-HCl buffer, at pH 7.5, and 10 mM beta-mercaptoethanol, and these proteins were separated by molecular weight.

SDS-acrylamide gel electrophoresis was performed to collect fractions corresponding to the pET32a-hNDST3ST (569-873aa) protein. The results are shown in FIG. 3.

To cleave His tag and Trx tag at the N-terminal of the collected proteins, the proteins were treated with thrombin protease (treatment with 1 unit of thrombin protease per 1 mg of target protein) and were reacted at 4° C. for 18 hours. Then, SDS-polyacrylamide gel electrophoresis was performed. The results of pET32a-hNDST3ST (569-873aa) are shown in FIG. 3.

### 7-4. Protein Denaturation Using Urea/Purification of Proteins by Column Chromatography

The pET32a-hNDST3ST (569-873aa) protein obtained at the step 7-3 was dissolved in a buffer solution containing 200 mM NaCl, 20 mM Hepes, at pH 7.4, and 8 M urea, and rotated at room temperature for 18 hours to ensure sufficient exposure to urea.

The His tag and Trx tag cleaved pET32a-hNDST3ST (569-873aa) protein was flowed through a nickel (Ni-NTA) column (GE Healthcare Life Sciences, USA) pre-equilibrated with the above-mentioned buffer solution again at room temperature, the flow-through was taken at a constant volume of aliquots. The proteins were eluted from the column using a 500 mM imidazole concentration gradient. 15% SDS-polyacrylamide gel electrophoresis was performed. The results for pET32a-hNDST3ST (569-873aa) are shown in FIG. 5C.

### Example 8. Expression of hNDST4ΔTMDA (72-569Aa) Recombinant Gene in E. coli

The expression vector pET32a-hNDST4ΔTMDA (72-569aa) obtained at the above-mentioned step was used to transform the expression host, E. coli BL21 (RIL) by the conventional methods. E. coli strains transformed with pET32a-hNDST4ΔTMDA (72-569aa) were cultured in 5.5 ml of LB broth containing 100 μg/mL of ampicillin at 37° C. for 16 hours. Then, 50 μl of the culture was subcultured in 5.5 ml of LB broth (containing 100 μg/mL of ampicillin). The culture was incubated at 37° C. until the absorbance value of the culture at 600 nm reached approximately 0.2-0.3. Then, the culture temperature was lowered to 18° C., and the culture was incubated until the absorbance value of the culture reached approximately 0.6. Then, IPTG (isopropyl-ß-D-galactopyranoside) was added thereto at a final concentration of 0.5 mM.

Cells incubated for 18 hours to which IPTG was previously added were centrifuged to collect the respective cell precipitates. The collected precipitates were each suspended in 0.5 ml of cell lysis buffer (200 mM sodium chloride, 20 mM Hepes-HCl buffer, pH 7.5, 5 mM beta-mercaptoethanol), and the cells were disrupted using a mini ultrasonicator (Sonic Dismembrator, Fisher, USA) in ice-cold water. The obtained solution was centrifuged at 13,000 rpm for 10 minutes in a centrifuge to separate the supernatant and the precipitate, which was subjected to 15% SDS-polyacrylamide gel electrophoresis, and analyzed by staining the protein pET32a-hNDST4ΔTMDA (72-569aa) with Coomassie Brilliant Blue (Bio-Rad 161-0400). The results for pET32a-hNDST4ΔTMDA (72-569aa) are shown in FIG. 4 (M: protein marker, I−: before IPTG induction, I+: cell lysate sample after IPTG induction, S: supernatant after centrifugation of IPTG-induced sample, P: precipitate after centrifugation of IPTG-induced sample).

### Example 9. Purification of Recombinant Protein hNDST4ΔTMDA (72-569Aa) in E. coli

9-1. Culturing and Disruption of E. coli Cells

In the way of Example 2, E. coli cells expressing the recombinant protein were cultured in 8 l quantities, these cells were centrifuged at 3,600 rpm for 20 minutes using a centrifuge to collect the E. coli cell precipitate. Then, this precipitate was suspended in a buffer solution containing 400 mM sodium chloride, 20 mM Hepes-HCl buffer, at pH 7.5, and 10 mM beta-mercaptoethanol at 15 ml per liter of cells, and the cells were disrupted using an ultrasonicator (Fisher, USA) in ice-cold water. The obtained solution was centrifuged at 18,000 rpm for 1 hour using a centrifuge to obtain the supernatant.

### 9-2. Purification by Column Chromatography

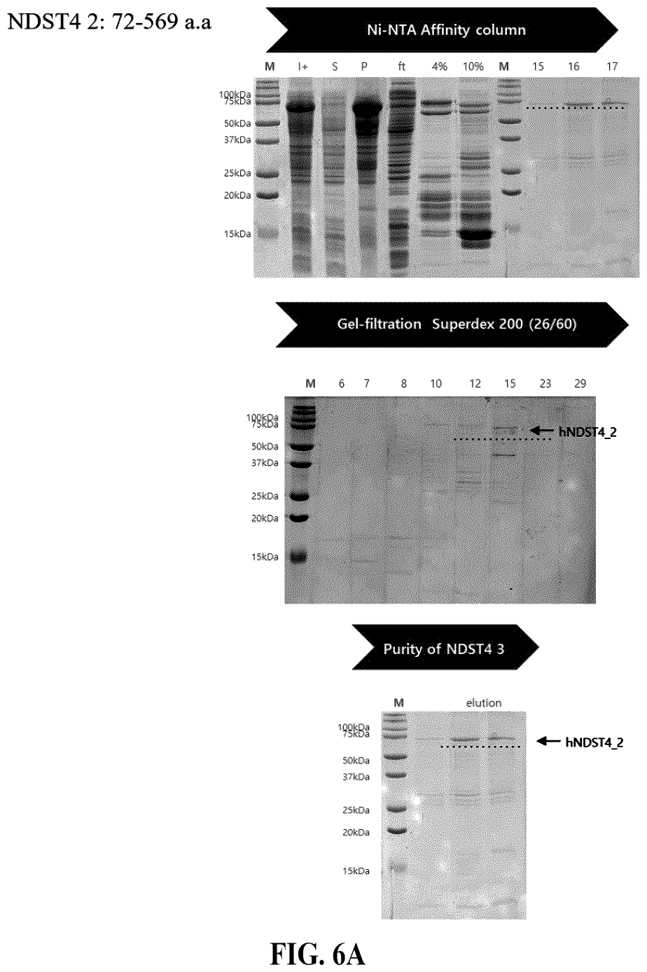

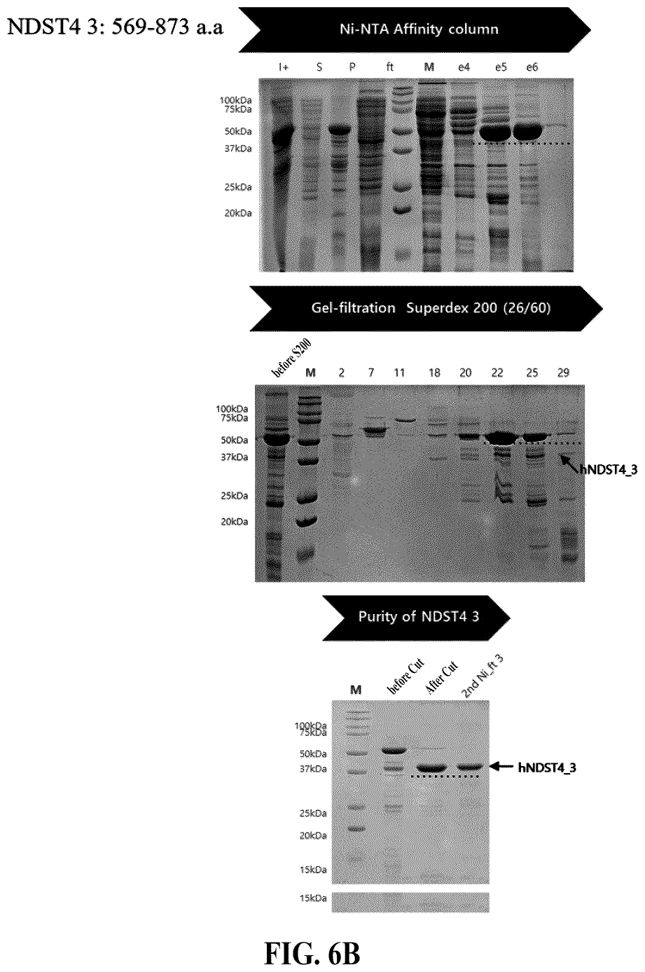

The supernatant of pET32a-hNDST4ΔTMDA (72-569aa) obtained at the step 1 was run on a nickel (Ni-NTA) column (GE Healthcare Life Sciences, USA) pre-equilibrated with the above-mentioned buffer solution under the condition of 100 mM NaCl, and the proteins were eluted from the column with a 500 mM imidazole concentration gradient using the above-mentioned buffer as the eluent. Each protein-containing fraction was identified by SDS-polyacrylamide gel electrophoresis, and the fractions were pooled. The results for pET32a-hNDST4ΔTMDA (72-569aa) are shown in FIG. 6A (M: protein marker, I−: before IPTG induction, I+: cell lysate sample after IPTG induction, S: supernatant after centrifugation of IPTG-induced sample, P: precipitate after centrifugation of IPTG-induced sample).

### Example 10. Expression of Recombinant hNDST4ST (569-873Aa) Gene in E. coli

The expression vector pET32a-hNDST4ST (569-873aa) obtained at the above-mentioned step was used to transform the expression host, E. coli BL21 (RIL) by conventional methods. E. coli strains transformed with pET32a-hNDST4ST (569-873aa) were cultured in 5.5 ml of LB broth containing 100 μg/mL of ampicillin at 37° C. for 16 hours. Then, 50 μl of the culture was subcultured in 5.5 ml of LB broth (containing 100 μg/mL of ampicillin). The culture was incubated at 37° C. until the absorbance value of the culture at 600 nm reached approximately 0.2-0.3. Then, the culture temperature was lowered to 18° C., and the culture was incubated until the absorbance value of the culture reached approximately 0.6. Then, IPTG (isopropyl-ß-D-galactopyranoside) was added thereto at a final concentration of 0.5 mM.

Cells incubated for 18 hours to which IPTG was previously added were centrifuged to collect the respective cell precipitates. The collected precipitates were each suspended in 0.5 ml of cell lysis buffer (200 mM sodium chloride, 20 mM Hepes-HCl buffer, at pH 7.5, 5 mM beta-mercaptoethanol), and the cells were disrupted using a mini ultrasonicator (Sonic Dismembrator, Fisher, USA) in ice-cold water. The obtained solution was centrifuged at 13,000 rpm for 10 minutes in a centrifuge to separate the supernatant and the precipitate, which was subjected to 15% SDS-polyacrylamide gel electrophoresis, and analyzed by staining the protein GST-ZnF_hUSP3 with Coomassie Brilliant Blue (Bio-Rad 161-0400). The results for pET32a-hNDST4 are shown in FIG. 4 (M: protein marker, I−: before IPTG induction, I+: cell lysate sample after IPTG induction, S: supernatant after centrifugation of IPTG-induced sample, P: precipitate after centrifugation of IPTG-induced sample).

### Example 11. Purification of Recombinant Protein hNDST4ST (569-873Aa) in E. coli

11-1. Culturing and Disruption of E. coli Cells

In the way of Example 2, E. coli cells expressing the recombinant protein were cultured in 8 l quantities, these cells were centrifuged at 3,600 rpm for 20 minutes using a centrifuge to collect the E. coli cell precipitate. Then, this precipitate was suspended in a buffer solution containing 400 mM sodium chloride, 20 mM Hepes-HCl buffer, at pH 7.5, 10 mM beta-mercaptoethanol and 5% glycerol at 27 ml per liter of cells, and the cells were disrupted using an ultrasonicator (Fisher, USA) in ice-cold water. The obtained solution was centrifuged at 18,000 rpm for 1 hour using a centrifuge to obtain the supernatant.

### 11-2. Purification by Column Chromatography

The supernatant of pET32a-hNDST4ST (569-873aa) obtained in Example 11-1 was run on a nickel (Ni-NTA) column (GE Healthcare Life Sciences, USA) pre-equilibrated with the above-mentioned buffer solution, and the proteins were eluted from the column with a 500 mM imidazole concentration gradient using the above-mentioned buffer as the eluent. Each protein-containing fraction was identified by SDS-polyacrylamide gel electrophoresis, and the fractions were pooled. The results for pET32a-hNDST4ST (569-873aa) are shown in FIG. 6B (M: protein marker, I−: before IPTG induction, I+: cell lysate sample after IPTG induction, S: supernatant after centrifugation of IPTG-induced sample, P: precipitate after centrifugation of IPTG-induced sample).

### 11-3. Separation and Purification of Proteins by Column Chromatography

The volume of the pET32a-hNDST4ST (569-873aa) protein obtained in Example 11-2 was concentrated to about 5 ml, and this volume of protein was injected into a gel permeation column (Superdex 200, 26/60 GL, GE Healthcare Life Sciences, USA) pre-equilibrated with the above-mentioned buffer solution. Then, the proteins were eluted with a buffer solution containing 400 mM sodium chloride, 20 mM Hepes-HCl buffer, at pH 7.5, 10 mM beta-mercaptoethanol, 5% glycerol, and 2 mM TCEP (Tris[2-carboxyethyl]phosphine hydrochloride), and these proteins were separated by molecular weight.

15% SDS-acrylamide gel electrophoresis was performed to collect fractions corresponding to the pET32a-hNDST4ST (569-873aa) protein.

To cleave His tag and Trx tag at the N-terminal of the collected proteins, the proteins were treated with thrombin protease (treatment with 1 unit of thrombin protease per 1 mg of target protein) and were reacted at 4° C. for 3 days. Then, SDS-polyacrylamide gel electrophoresis was performed. The results for pET32a-hNDST4ST (569-873aa) are shown in FIG. 6B.

### 11-4. Protein Denaturation Using Urea/Purification of Proteins by Column Chromatography

Since the pET32a-hNDST4ST (569-873aa) protein obtained in Example 11-2 was in a precipitated state, to dissolve it back into a liquid state, it was washed concentration-wise with 1 M, 2 M, and 4 M Urea and the precipitates were dissolved in 8 M Urea again. Afterwards, the dissolved precipitates were flowed through a nickel (Ni-NTA) column (GE Healthcare Life Sciences, USA) pre-equilibrated with a buffer solution containing 200 mM sodium chloride, 20 mM HEPES-HCl buffer, at pH 7.5, and 8 M urea. The flow-through was taken in 3 ml aliquots and subjected to SDS-acrylamide gel electrophoresis to confirm that the pET32a-hNDST4 protein was purified. The results for pET32a-hNDST4ST (569-873aa) are shown in FIG. 6B.

### Example 12. Evaluation of Enzymatic Activity of Human NDST3 and NDST4 Variants In Vitro

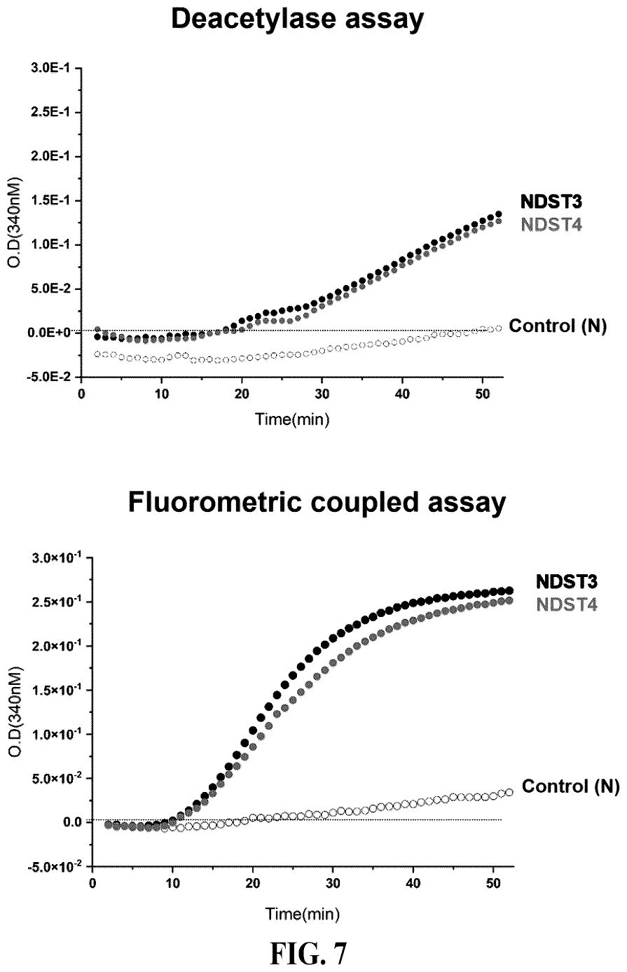

To determine the enzymatic activity of NDST3 (hNDST3ΔTMDA, hNDST3ST) and NDST4 (hNDST4ΔTMDA, hNDST4ST) cleaved from polysaccharide substrates, 250 nM NDST3 variants (hNDST3ΔTMDA, hNDST3ST) and NDST4 variants (hNDST4ΔTMDA, hNDST4ST), 350 nM Sult1a1-K65E-R68G, 20 μM PAPS, and 4 mM 4MU-sulfate were reacted in 50 mM Tris-HCl, at pH 7.5, 15 mM MgCl₂, and 1 mM DTT, under different concentrations of K5 or HS polysaccharides (low-sulfated fraction; Iduron, GAG HS-I). The controls without polysaccharides were excluded from the reaction prior to further analysis. To measure pseudo-first-order activity for PAPS, 2-fold dilutions of PAPS from 50 μM, 0.5 mg/mL HS, NDST3 variants (hNDST3ΔTMDA, hNDST3ST) and NDST4 variants (hNDST4ΔTMDA, hNDST4ST), 350 nM Sult1a1-K65E-R68G and 4 mM 4Mu-sulfate were reacted in 50 mM Tris-HCl, at pH 7.5, 15 mM MgCl₂, and 1 mM DTT. The controls without PAPS were excluded from the reaction prior to further analysis. All reactions were performed in black half-area 96-well plates (Greiner) in a total reaction volume of 20 μL. All reactions were initiated by the addition of polysaccharides, and the changes in fluorescence were recorded with a Clariostar plate reader (SpectraMax® M3, Molecular Devices) using the 4MU preset. An 8 point 2-fold serial dilution standard curve of 4MU (from 10 μM) in 50 mM Tris-HCl at pH 7, 15 mM MgCl₂ was used to calculate the evolution in 4MU during the reaction. The results are shown in FIG. 7.

Although a number of embodiments have been described with reference to limited drawings, one of ordinary skill in the art will recognize that various modifications and alterations may be made to these embodiments based on the above detailed description. For example, suitable results may be achieved if the described techniques are performed in a different order, and/or if components in a described system, architecture, device, or circuit are combined in a different manner, and/or replaced or supplemented by other components or their equivalents.

Therefore, other implementations, other examples, and equivalents to the claims are also within the scope of the following claims.

What is claimed is:

1. A peptide having deacetylase activity,
wherein the peptide comprises an amino acid sequence of SEQ ID NO: 3 or SEQ ID NO: 7.

2. A method for preparing the peptide according to claim 1, comprising the steps of:
producing an expression vector comprising a gene encoding the peptide of claim 1;
transforming E. coli with the expression vector;
culturing the transformed E. coli;
disrupting and centrifuging the transformed E. coli to obtain a cell precipitate; and
purifying the peptide according to claim 1 from the cell precipitate.

3. A peptide having sulfotransferase activity,
wherein the peptide comprises an amino acid sequence of SEQ ID NO: 4 or SEQ ID NO: 8.

4. A method for preparing the peptide according to claim 3, comprising the steps of:
producing an expression vector comprising a gene encoding the peptide of claim 3;
transforming E. coli with the expression vector;
culturing the transformed E. coli;
disrupting and centrifuging the transformed E. coli to obtain a cell precipitate; and
purifying the peptide according to claim 3 from the cell precipitate.


SEQUENCE LISTING (26 sequences)

SEQ ID NO: 1 - protein, 873 aa, Homo sapiens
  source 1..873: note = T0 = Human N-Deacetylase And N-Sulfotransferase 3 (1-873 a.a)
MSFIMKLHRHFQRTVILLATFCMVSIIISAYYLYSGYKQENELSETASEVDCGDLQHLPYQLMEVKAMKLFDASRTDPTVLVFVESQYSSLGQDIIMILESSRFQYHIEIAPGKGDLPVLIDKMKGKYILIIYENILKYINMDSWNRSLLDKYCVEYGVGVIGFHKTSEKSVQSFQLKGFPFSIYGNLAVKDCCINPHSPLIRVTKSSKLEKGSLPGTDWTVFQINHSAYQPVIFAKVKTPENLSPSISKGAFYATIIHDLGLHDGIQRVLFGNNLNFWLHKLIFIDAISFLSGKRLTLSLDRYILVDIDDIFVGKEGTRMNTNDVKALLDTQNLLRAQITNFTFNLGFSGKFYHTGTEEEDEGDDCLLGSVDEFWWFPHMWSHMQPHLFHNESSLVEQMILNKKFALEHGIPTDMGYAVAPHHSGVYPVHVQLYEAWKKVWNIKITSTEEYPHLKPARYRRGFIHKNIMVLPRQTCGLFTHTIFYKEYPGGPKELDKSIQGGELFFTVVLNPISIFMTHLSNYGNDRLGLYTFVNLANFVKSWTNLRLQTLPPVQLAHKYFELFPDQKDPLWQNPCDDKRHRDIWSKEKTCDRLPKFLVIGPQKTGTTALYLFLVMHPSILSNSPSPKTFEEVQFFNRNNYHRGIDWYMDFFPVPSNVTTDFLFEKSANYFHSEEAPKRAASLVPKAKIITILIDPSDRAYSWYQHQRSHEDPAALKFSFYEVISAGPRAPSELRALQKRCLVPGWYASHIERWLVYFPPFQLLIIDGQQLRTDPATVMDEVQKFLGVLPHYNYSEALTFDSHKGFWCQLLEEGKTKCLGKSKGRKYPPMDSDSRTFLSSYYRDHNVELSKLLHKLGQPLPSWLRQELQKVR

SEQ ID NO: 2 - protein, 802 aa, synthetic construct
  REGION 1..802: note = T1 = deltaTM_Human N-Deacetylase And N-Sulfotransferase 3 (72 - 873 a.a)
DASRTDPTVLVFVESQYSSLGQDIIMILESSRFQYHIEIAPGKGDLPVLIDKMKGKYILIIYENILKYINMDSWNRSLLDKYCVEYGVGVIGFHKTSEKSVQSFQLKGFPFSIYGNLAVKDCCINPHSPLIRVTKSSKLEKGSLPGTDWTVFQINHSAYQPVIFAKVKTPENLSPSISKGAFYATIIHDLGLHDGIQRVLFGNNLNFWLHKLIFIDAISFLSGKRLTLSLDRYILVDIDDIFVGKEGTRMNTNDVKALLDTQNLLRAQITNFTFNLGFSGKFYHTGTEEEDEGDDCLLGSVDEFWWFPHMWSHMQPHLFHNESSLVEQMILNKKFALEHGIPTDMGYAVAPHHSGVYPVHVQLYEAWKKVWNIKITSTEEYPHLKPARYRRGFIHKNIMVLPRQTCGLFTHTIFYKEYPGGPKELDKSIQGGELFFTVVLNPISIFMTHLSNYGNDRLGLYTFVNLANFVKSWTNLRLQTLPPVQLAHKYFELFPDQKDPLWQNPCDDKRHRDIWSKEKTCDRLPKFLVIGPQKTGTTALYLFLVMHPSILSNSPSPKTFEEVQFFNRNNYHRGIDWYMDFFPVPSNVTTDFLFEKSANYFHSEEAPKRAASLVPKAKIITILIDPSDRAYSWYQHQRSHEDPAALKFSFYEVISAGPRAPSELRALQKRCLVPGWYASHIERWLVYFPPFQLLIIDGQQLRTDPATVMDEVQKFLGVLPHYNYSEALTFDSHKGFWCQLLEEGKTKCLGKSKGRKYPPMDSDSRTFLSSYYRDHNVELSKLLHKLGQPLPSWLRQELQKVR

SEQ ID NO: 3 - protein, 498 aa, synthetic construct
  REGION 1..498: note = T2 = Human NDST3_N-Deacetylase domain (72 - 569 a.a)
DASRTDPTVLVFVESQYSSLGQDIIMILESSRFQYHIEIAPGKGDLPVLIDKMKGKYILIIYENILKYINMDSWNRSLLDKYCVEYGVGVIGFHKTSEKSVQSFQLKGFPFSIYGNLAVKDCCINPHSPLIRVTKSSKLEKGSLPGTDWTVFQINHSAYQPVIFAKVKTPENLSPSISKGAFYATIIHDLGLHDGIQRVLFGNNLNFWLHKLIFIDAISFLSGKRLTLSLDRYILVDIDDIFVGKEGTRMNTNDVKALLDTQNLLRAQITNFTFNLGFSGKFYHTGTEEEDEGDDCLLGSVDEFWWFPHMWSHMQPHLFHNESSLVEQMILNKKFALEHGIPTDMGYAVAPHHSGVYPVHVQLYEAWKKVWNIKITSTEEYPHLKPARYRRGFIHKNIMVLPRQTCGLFTHTIFYKEYPGGPKELDKSIQGGELFFTVVLNPISIFMTHLSNYGNDRLGLYTFVNLANFVKSWTNLRLQTLPPVQLAHKYFELFPDQK

SEQ ID NO: 4 - protein, 305 aa, synthetic construct
  REGION 1..305: note = T3 = Human NDST3_ N-Sulfotransferase domain (569 ? 873 a.a)
KDPLWQNPCDDKRHRDIWSKEKTCDRLPKFLVIGPQKTGTTALYLFLVMHPSILSNSPSPKTFEEVQFFNRNNYHRGIDWYMDFFPVPSNVTTDFLFEKSANYFHSEEAPKRAASLVPKAKIITILIDPSDRAYSWYQHQRSHEDPAALKFSFYEVISAGPRAPSELRALQKRCLVPGWYASHIERWLVYFPPFQLLIIDGQQLRTDPATVMDEVQKFLGVLPHYNYSEALTFDSHKGFWCQLLEEGKTKCLGKSKGRKYPPMDSDSRTFLSSYYRDHNVELSKLLHKLGQPLPSWLRQELQKVR

SEQ ID NO: 5 - protein, 872 aa, synthetic construct
  REGION 1..872: note = T4= Human N-Deacetylase And N-Sulfotransferase 4 (1-873 a.a)
MNLIVKLRRSFRTLIVLLATFCLVSIVISAYFLYSGYKQEMTLIETTAEAECTDIKILPYRSMELKTVKPIDTSKTDPTVLLFVESQYSQLGQDIIAILESSRFQYHMVIAPGKGDIPPLTDNGKGKYTLVIYENILKYVSMDSWNRELLEKYCVEYSVSIIGFHKANENSLPSTQLKGFPLNLFNNLALKDCFVNPQSPLLHITKAPKVEKGPLPGEDWTIFQYNHSTYQPVLLTELQTEKSLSSLSSKTLFATVIQDLGLHDGIQRVLFGNNLNFWLHKLIFIDAISFLSGKRLTLSLDRYILVDIDDIFVGKEGTRMNVKDVKALLETQNLLRTQVANFTFNLGFSGKFYHTGTEEEDEGDDLLLRSVDEFWWFPHMWSHMQPHLFHNESSLVEQMILNKEFALEHGIPINMGYAVAPHHSGVYPVHIQLYAAWKKVWGIQVTSTEEYPHLKPARYRKGFIHNSIMVLPRQTCGLFTHTIFYKEYPGGPQELDKSIRGGELFLTILLNPISIFMTHLSNYGNDRLGLYTFVNLVNFVQSWTNLKLQTLPPVQLAHQYFELFPEQKDPLWQNPCDDKRHKDIWSREKTCDHLPKFLVIGPQKTGTTALYLFLLMHPSIISNLPSPKTFEEVQFFNGNNYHKGIDWYMDFFPTPSNTTSDFLFEKSANYFHSEEAPRRAASLVPKAKIITILIDPSDRAYSWYQHQRSHEDPAALRFNFYEVISTGHWAPSDLKTLQRRCLVPGWYAVHIERWLTYFATSQLLIIDGQQLRSDPATVMDEVQKFLGVTPRYNYSEALTFDPQKGFWCQLLEGGKTKCLGKSKGRKYPPMDPESRTFLSNYYRDHNVELSKLLHRLGQPLPSWLRQELQKVR

SEQ ID NO: 6 - protein, 801 aa, synthetic construct
  REGION 1..801: note = T5= deltaTM_Human N-Deacetylase And N-Sulfotransferase 4 (72 - 873 a.a)
DTSKTDPTVLLFVESQYSQLGQDIIAILESSRFQYHMVIAPGKGDIPPLTDNGKGKYTLVIYENILKYVSMDSWNRELLEKYCVEYSVSIIGFHKANENSLPSTQLKGFPLNLFNNLALKDCFVNPQSPLLHITKAPKVEKGPLPGEDWTIFQYNHSTYQPVLLTELQTEKSLSSLSSKTLFATVIQDLGLHDGIQRVLFGNNLNFWLHKLIFIDAISFLSGKRLTLSLDRYILVDIDDIFVGKEGTRMNVKDVKALLETQNLLRTQVANFTFNLGFSGKFYHTGTEEEDEGDDLLLRSVDEFWWFPHMWSHMQPHLFHNESSLVEQMILNKEFALEHGIPINMGYAVAPHHSGVYPVHIQLYAAWKKVWGIQVTSTEEYPHLKPARYRKGFIHNSIMVLPRQTCGLFTHTIFYKEYPGGPQELDKSIRGGELFLTILLNPISIFMTHLSNYGNDRLGLYTFVNLVNFVQSWTNLKLQTLPPVQLAHQYFELFPEQKDPLWQNPCDDKRHKDIWSREKTCDHLPKFLVIGPQKTGTTALYLFLLMHPSIISNLPSPKTFEEVQFFNGNNYHKGIDWYMDFFPTPSNTTSDFLFEKSANYFHSEEAPRRAASLVPKAKIITILIDPSDRAYSWYQHQRSHEDPAALRFNFYEVISTGHWAPSDLKTLQRRCLVPGWYAVHIERWLTYFATSQLLIIDGQQLRSDPATVMDEVQKFLGVTPRYNYSEALTFDPQKGFWCQLLEGGKTKCLGKSKGRKYPPMDPESRTFLSNYYRDHNVELSKLLHRLGQPLPSWLRQELQKVR

SEQ ID NO: 7 - protein, 497 aa, synthetic construct
  REGION 1..497: note = T6= Human NDST4_N-Deacetylase domain (72 - 569 a.a)
DTSKTDPTVLLFVESQYSQLGQDIIAILESSRFQYHMVIAPGKGDIPPLTDNGKGKYTLVIYENILKYVSMDSWNRELLEKYCVEYSVSIIGFHKANENSLPSTQLKGFPLNLFNNLALKDCFVNPQSPLLHITKAPKVEKGPLPGEDWTIFQYNHSTYQPVLLTELQTEKSLSSLSSKTLFATVIQDLGLHDGIQRVLFGNNLNFWLHKLIFIDAISFLSGKRLTLSLDRYILVDIDDIFVGKEGTRMNVKDVKALLETQNLLRTQVANFTFNLGFSGKFYHTGTEEEDEGDDLLLRSVDEFWWFPHMWSHMQPHLFHNESSLVEQMILNKEFALEHGIPINMGYAVAPHHSGVYPVHIQLYAAWKKVWGIQVTSTEEYPHLKPARYRKGFIHNSIMVLPRQTCGLFTHTIFYKEYPGGPQELDKSIRGGELFLTILLNPISIFMTHLSNYGNDRLGLYTFVNLVNFVQSWTNLKLQTLPPVQLAHQYFELFPEQK

SEQ ID NO: 8 - protein, 305 aa, synthetic construct
  REGION 1..305: note = T3 = Human NDST4_ N-Sulfotransferase domain (569 - 873 a.a)
KDPLWQNPCDDKRHKDIWSREKTCDHLPKFLVIGPQKTGTTALYLFLLMHPSIISNLPSPKTFEEVQFFNGNNYHKGIDWYMDFFPTPSNTTSDFLFEKSANYFHSEEAPRRAASLVPKAKIITILIDPSDRAYSWYQHQRSHEDPAALRFNFYEVISTGHWAPSDLKTLQRRCLVPGWYAVHIERWLTYFATSQLLIIDGQQLRSDPATVMDEVQKFLGVTPRYNYSEALTFDPQKGFWCQLLEGGKTKCLGKSKGRKYPPMDPESRTFLSNYYRDHNVELSKLLHRLGQPLPSWLRQELQKVR

SEQ ID NO: 9 - DNA, 32 bp, synthetic construct
  misc_feature 1..32: note = HUMAN NDST3 full (1-873 a.a)_primer_F
  source 1..32: mol_type = other DNA
caccggatccatgagttttatcatgaagcttc

SEQ ID NO: 10 - DNA, 39 bp, synthetic construct
  misc_feature 1..39: note = HUMAN NDST3 full (1-873 a.a)_primer_R
  source 1..39: mol_type = other DNA
gctaggcatctcgagttactatcttactttctgcagctc

SEQ ID NO: 11 - DNA, 31 bp, synthetic construct
  misc_feature 1..31: note = HUMAN NDST3 deltaTM (72-873 a.a)_primer_F
  source 1..31: mol_type = other DNA
caccggatccatggatgcctcaaggacagac

SEQ ID NO: 12 - DNA, 39 bp, synthetic construct; identical to SEQ ID NO: 10
  misc_feature 1..39: note = HUMAN NDST3 deltaTM (72-873 a.a)_primer_R
  source 1..39: mol_type = other DNA

SEQ ID NO: 13 - DNA, 32 bp, synthetic construct; identical to SEQ ID NO: 9
  misc_feature 1..32: note = HUMAN NDST3 N-Deacetylase (1-569 a.a)_Primer_F
  source 1..32: mol_type = other DNA

SEQ ID NO: 14 - DNA, 40 bp, synthetic construct
  misc_feature 1..40: note = HUMAN NDST3 N-Deacetylase (1-569 a.a)_Primer_R
  source 1..40: mol_type = other DNA
gctaggcatctcgagttactatttctgatcaggaaacagc

SEQ ID NO: 15 - DNA, 31 bp, synthetic construct; identical to SEQ ID NO: 11
  misc_feature 1..31: note = HUMAN NDST3 deltaTM N-Deacetylase (72-569 a.a)_Primer_F
  source 1..31: mol_type = other DNA

SEQ ID NO: 16 - DNA, 40 bp, synthetic construct; identical to SEQ ID NO: 14
  misc_feature 1..40: note = HUMAN NDST3 deltaTM N-Deacetylase (72-569 a.a)_Primer_R
  source 1..40: mol_type = other DNA

SEQ ID NO: 17 - DNA, 33 bp, synthetic construct
  misc_feature 1..33: note = HUMAN NDST3 N-Sulfotransferase (569-873 a.a)_Primer_F
  source 1..33: mol_type = other DNA
caccggatccatggaccctctctggcagaatcc

SEQ ID NO: 18 - DNA, 39 bp, synthetic construct; identical to SEQ ID NO: 10
  misc_feature 1..39: note = HUMAN NDST3 N-Sulfotransferase (569-873 a.a)_Primer_R
  source 1..39: mol_type = other DNA

SEQ ID NO: 19 - DNA, 27 bp, synthetic construct
  misc_feature 1..27: note = HUMAN NDST4 Full (1-873 a.a)_Primer_F
  source 1..27: mol_type = other DNA
caccggatccatgaatcttattgtgaa

SEQ ID NO: 20 - DNA, 37 bp, synthetic construct
  misc_feature 1..37: note = HUMAN NDST4 Full (1-873 a.a)_Primer_R
  source 1..37: mol_type = other DNA
gctaggcatctcgagttatctcactttctgcagttcc

SEQ ID NO: 21 - DNA, 29 bp, synthetic construct
  misc_feature 1..29: note = HUMAN NDST4 deltaTM (72-873 a.a)_Primer_F
  source 1..29: mol_type = other DNA
caccggatccatggacacatccaaaacgg

SEQ ID NO: 22 - DNA, 37 bp, synthetic construct
  misc_feature 1..37: note = HUMAN NDST4 deltaTM (72-873 a.a)_Primer_R
  source 1..37: mol_type = other DNA
gctaggcatctcgagttagtctttctgctcagggaag

SEQ ID NO: 23 - DNA, 31 bp, synthetic construct; identical to SEQ ID NO: 11
  misc_feature 1..31: note = HUMAN NDST4 deltaTM N-Deacetylase (72-569 a.a)_Primer_F
  source 1..31: mol_type = other DNA

SEQ ID NO: 24 - DNA, 37 bp, synthetic construct; identical to SEQ ID NO: 22
  misc_feature 1..37: note = HUMAN NDST4 deltaTM N-Deacetylase (72-569 a.a)_Primer_R
  source 1..37: mol_type = other DNA

SEQ ID NO: 25 - DNA, 30 bp, synthetic construct
  misc_feature 1..30: note = HUMAN NDST4 N-Sulfotransferase (569-873 a.a)_Primer_F
  source 1..30: mol_type = other DNA
caccggatccatggaccctctatggcagaa

SEQ ID NO: 26 - DNA, 37 bp, synthetic construct; identical to SEQ ID NO: 20
  misc_feature 1..37: note = HUMAN NDST4 N-Sulfotransferase (569-873 a.a)_Primer_R
  source 1..37: mol_type = other DNA